(19) United States
(12) Patent Application Publication
(10) Pub. No.: US 2022/0111017 A1
(43) Pub. Date: Apr. 14, 2022

(54) IMMUNOMODULATORY COMPOSITIONS AND METHODS

(71) Applicant: Innate Biologics LLC, Harrisburg, PA (US)

(72) Inventors: Peter MONDICS, Newtown Square, PA (US); Jacob MANDELL, Bala Cynwyd, PA (US); Surbhi JOSHI, Yardley, PA (US)

(21) Appl. No.: 17/514,472

(22) Filed: Oct. 29, 2021

Related U.S. Application Data
(63) Continuation-in-part of application No. PCT/US2020/030958, filed on May 1, 2020.
(60) Provisional application No. 62/841,312, filed on May 1, 2019.

(51) Int. Cl. A61K 38/45; A61K 38/16; A61P 37/06; A61K 38/48

(52) U.S. Cl. CPC A61K 38/45; A61K 38/164; A61K 38/00; A61K 38/4886; A61P 37/06

(57) ABSTRACT

Provided herein are methods and compositions comprising constructs that include two or more truncated T3SS bacterial effector polypeptides. Also provided are pharmaceutical compositions comprising the constructs and methods of treatment of inflammatory disorders based on administering such constructs.

### RELATED APPLICATIONS AND INCORPORATION BY REFERENCE

This application is a continuation-in-part application of international patent application Serial No. PCT/US2020/030958 filed 1 May 2020, which published as PCT Publication No. WO 2020/223601 on 5 Nov. 2020, which claims priority under 35 U.S.C. § 119(e)(1) from U.S. Provisional Application Ser. No. 62/841,312, filed May 1, 2019.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted electronically in ASCII format and is hereby incorporated by reference in its entirety. Said ASCII copy, created on May 1, 2020, is named G6113_00029_SL.txt and is 60,849 bytes in size.

### FIELD OF THE INVENTION

The present invention relates to compositions and methods for use in treatment of inflammatory conditions.

### BACKGROUND OF THE INVENTION

Inflammation is a physiological defense mechanism for recognition and removal of potentially harmful stimuli, such as pathogens, irritants or damaged cells. Inflammation is classified as either acute or chronic. Acute inflammation refers to the body's immediate immune response to help prevent further injury and facilitate healing. Acute inflammation is typically self-limiting. Under some circumstances, the inflammatory process becomes continuous, resulting in the development of chronic inflammation. Chronic inflammation results in chronic pain, redness, swelling, stiffness, and damage to normal tissues. Chronic inflammation is associated with a wide range of disorders that have significant worldwide morbidity and mortality rates, for example, arthritis and joint diseases, cardiovascular diseases, allergies, chronic obstructive pulmonary disease, diabetes, inflammatory bowel disease, and cancer. There is a continuing need for new and effective methods of treating inflammatory conditions.

Citation or identification of any document in this application is not an admission that such document is available as prior art to the present invention.

### SUMMARY OF THE INVENTION

Disclosed herein are constructs comprising compositions two or more truncated T3SS bacterial effector polypeptides. The constructs provided herein can include two or more truncated T3SS bacterial effector polypeptides comprising a portion of the full length-bacterial effector polypeptides YopE, YopJ, YopM, NleE, NIeC, NleB, OspZ, IpaH4.5, IpaH7.8, and IpaH9.8. The constructs can further comprise a protein transduction domain. The constructs can be formulated as pharmaceuticals for use in the treatment of inflammatory conditions.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features and advantages of the present invention will be more fully disclosed in, or rendered obvious by, the following detailed description of the preferred embodiment of the invention, which is to be considered together with the accompanying drawings wherein like numbers refer to like parts and further wherein:

FIG. 1 is a listing of bacterial effector polypeptide amino acid and nucleotide sequences.

FIG. 2 is a diagram showing a construct comprising a YopE polypeptide and an OspZ polypeptide.

FIG. 3 is a diagram showing a construct comprising a truncated YopM polypeptide and a truncated OspZ polypeptide.

FIG. 4 is a diagram showing a construct comprising a truncated YopM polypeptide and a truncated NIeC polypeptide.

FIG. 5 is a diagram showing domains of the full-length IpaH9.8 polypeptide and the full-length IpaH4.5 polypeptide.

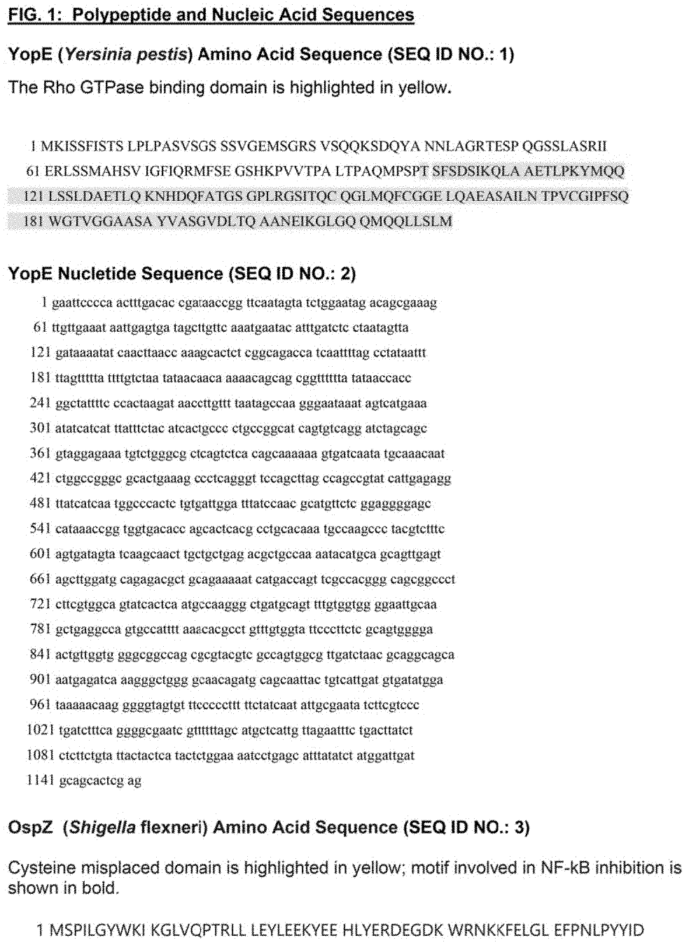

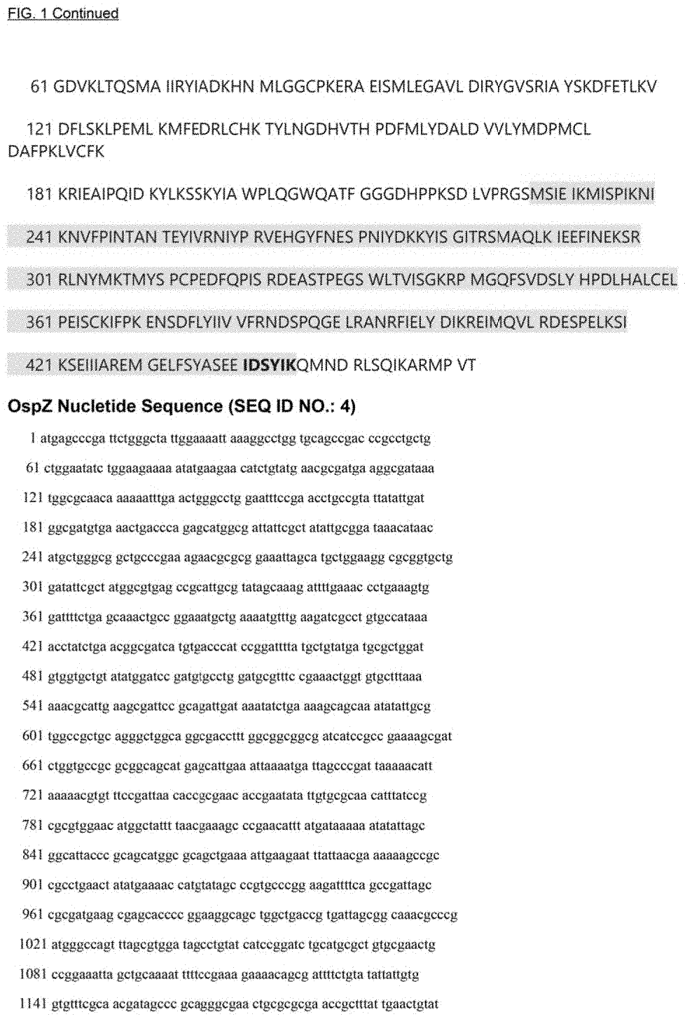

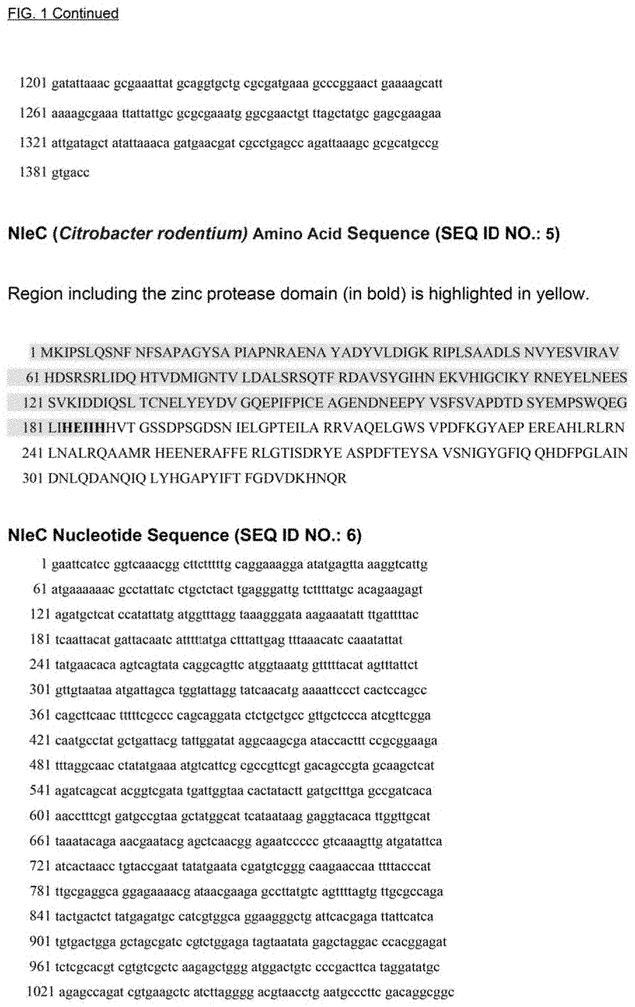

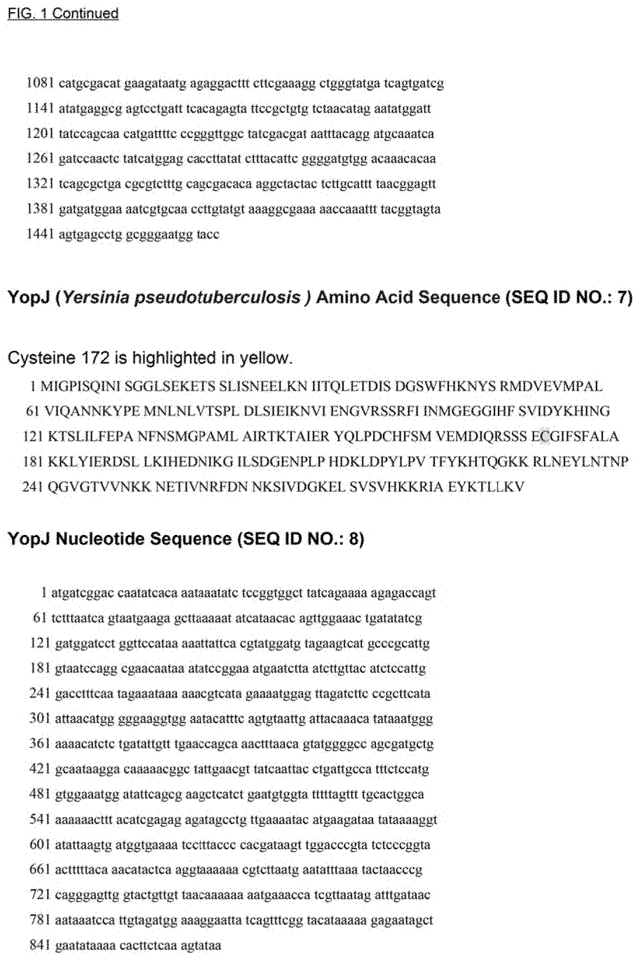

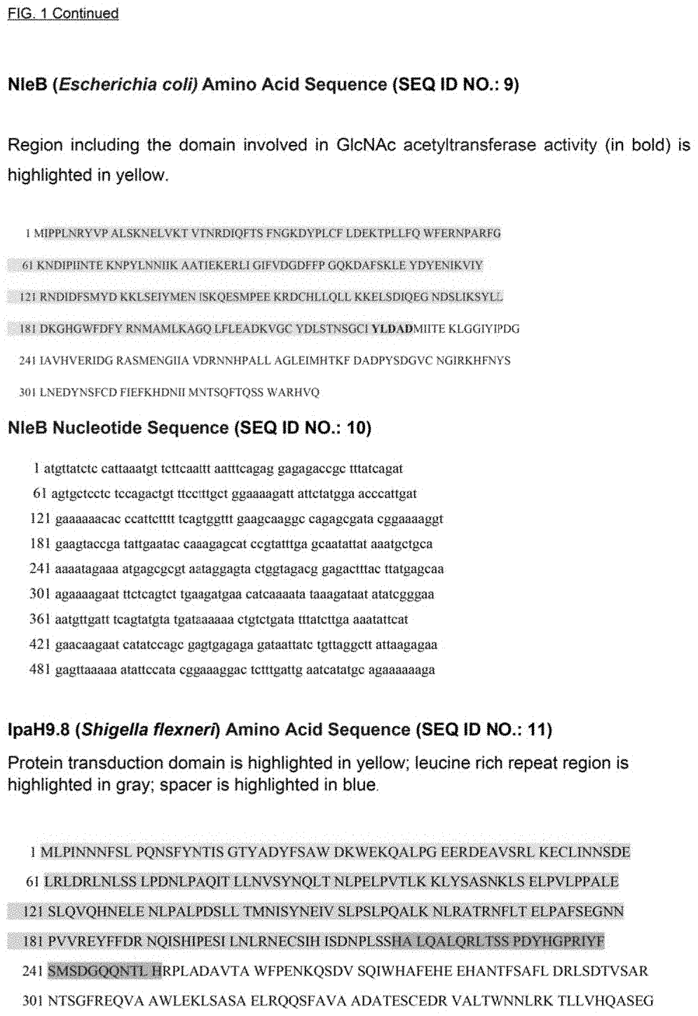

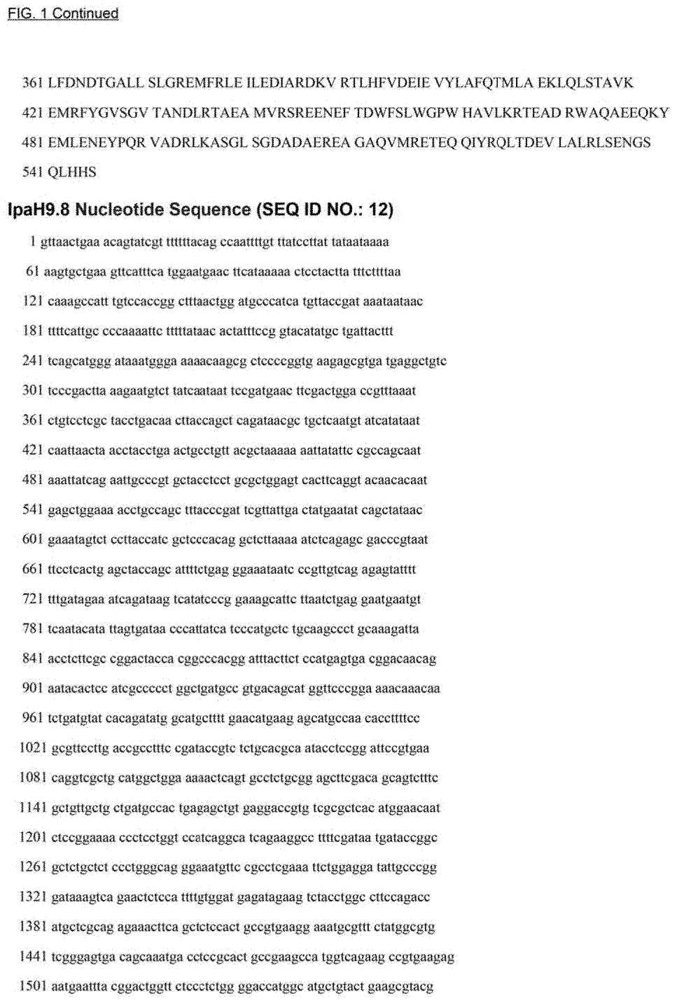

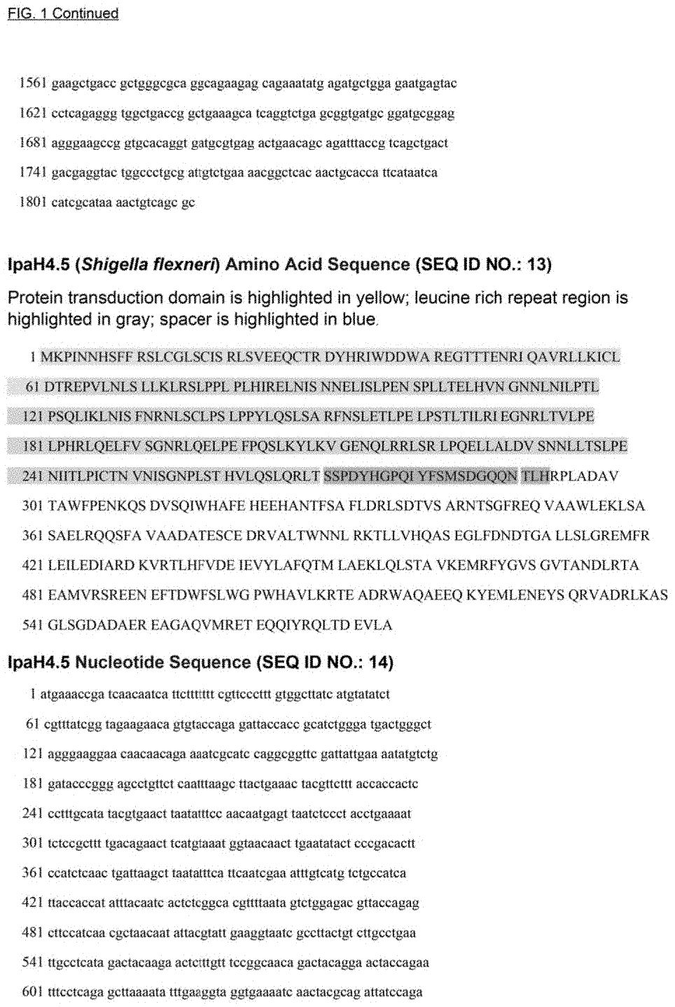

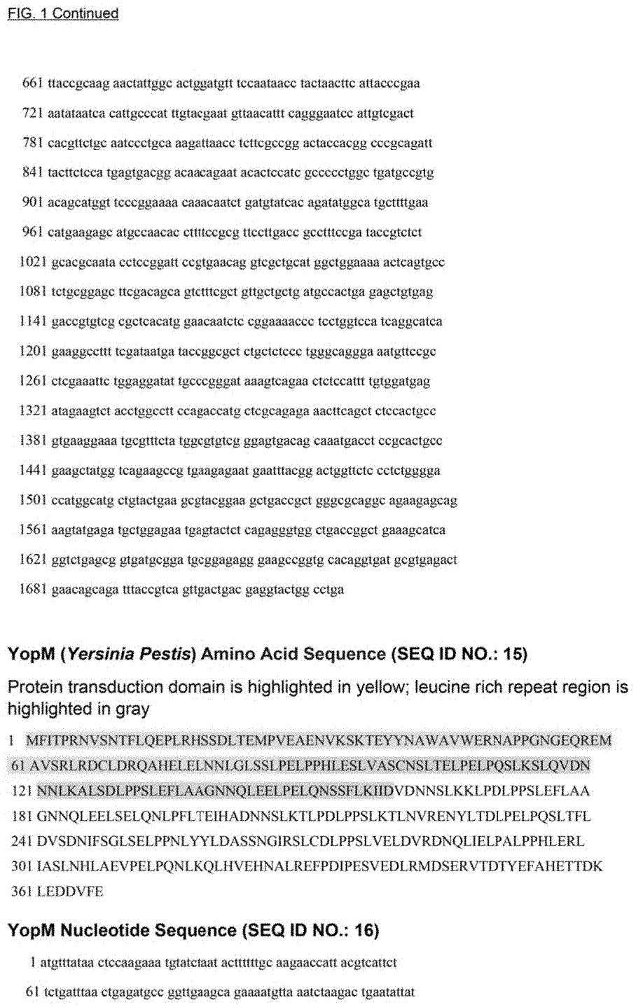

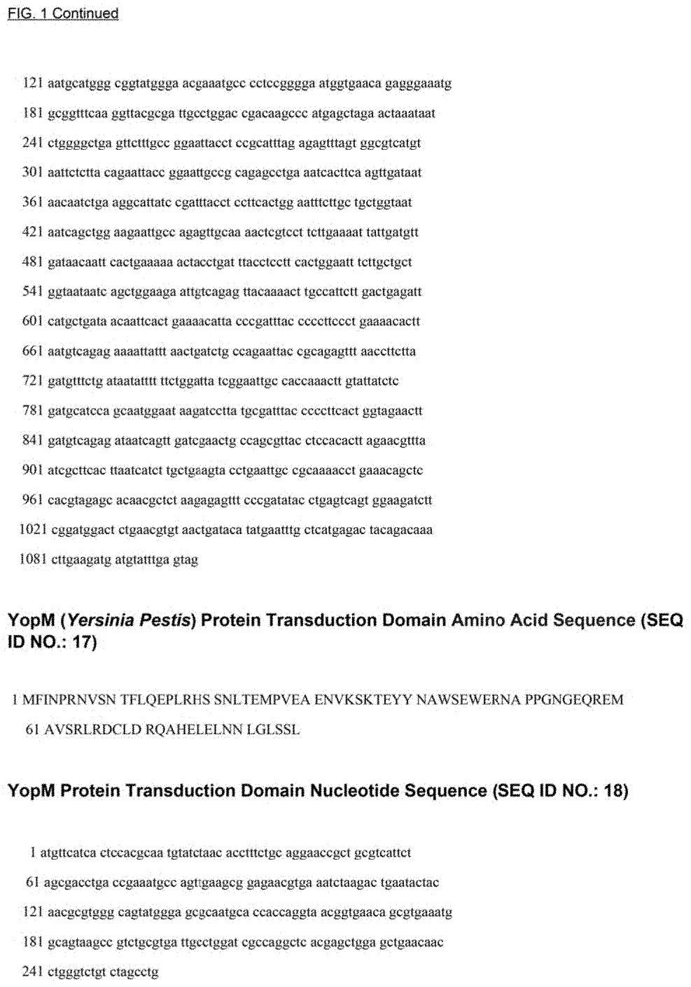

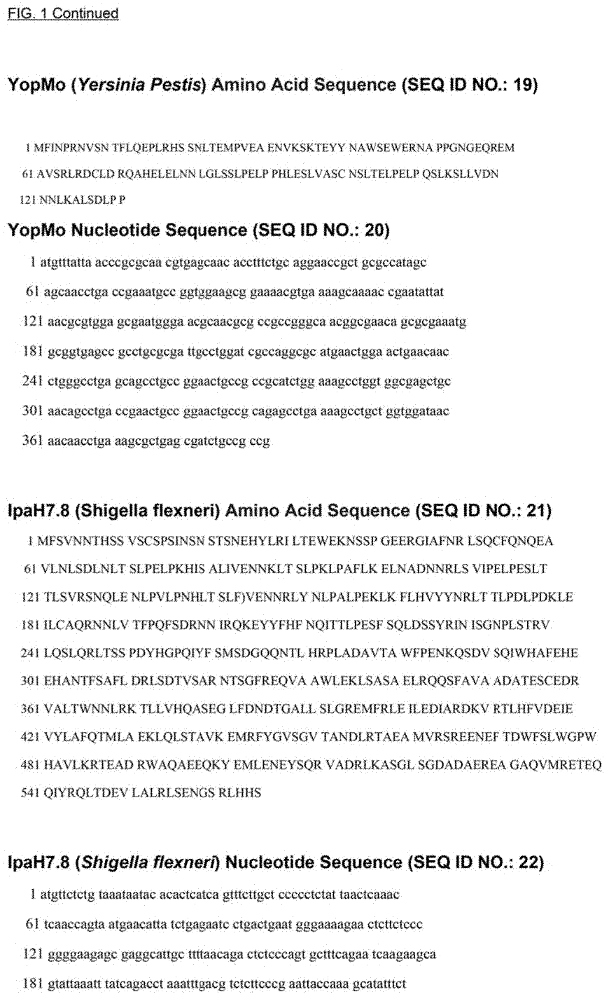

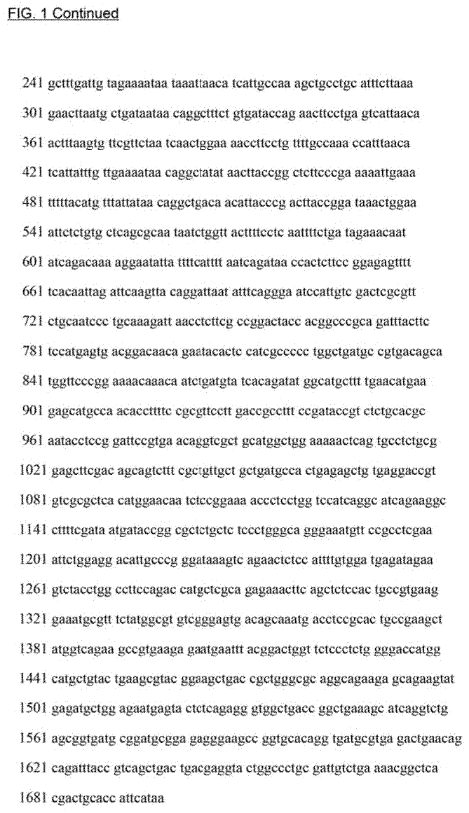

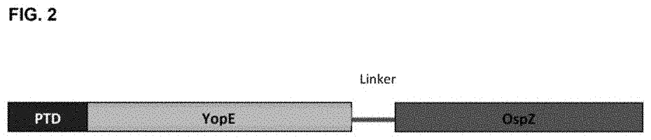

### DETAILED DESCRIPTION OF THE PREFERRED EMBODIMENT

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. When only a single machine is illustrated, the term “machine” shall also be taken to include any collection of machines that individually or jointly execute a set (or multiple sets) of instructions to perform any one or more of the methodologies discussed herein. In the claims, means-plus-function clauses, if used, are intended to cover the structures described, suggested, or rendered obvious by the written description or drawings for performing the recited function, including not only structural equivalents but also equivalent structures.

Disclosed herein are compositions and methods for treatment of an inflammatory condition. The compositions can include constructs comprising two or more truncated T3SS bacterial effector polypeptides. Bacterial effector polypeptides are injected into host cells, usually via a type III secretion system (T3SS) during the course of an infection. Such polypeptides inhibit or disable host immune responses by targeting host inflammatory signaling pathways, allowing the pathogen to undermine the host defense to ensure bacterial survival. The compositions and methods disclosed herein have immunomodulatory activity and are thus useful for the treatment of inflammatory conditions. The truncated T3SS bacterial effector polypeptides can provide enhanced pharmacokinetic properties and bioavailability to increase treatment efficacy.

The constructs provided herein can include two or more truncated T3SS bacterial effector polypeptides comprising a portion of a T3SS full length-bacterial effector polypeptide. T3SS full length-bacterial effector polypeptide can have biological activity such as E3 ubiquitin ligase activity, RhoGTPase modulatory activity, cysteine methylase activity, zinc metalloprotease activity, acetyltransferase activity or O-GlcNac transferase activity. The constructs provided herein can include two or more truncated T3SS bacterial effector polypeptides comprising a portion of the full length-bacterial effector polypeptides YopE, YopJ, YopM, NIeC, NleB, NleE, OspZ, IpaH4.5, IpaH7.8, and IpaH9.8. The two or more truncated T3SS bacterial effector polypeptides can be the same or they can be different. Exemplary constructs can include a truncated YopE polypeptide and a truncated OspZ polypeptide; a truncated YopM polypeptide and a truncated OspZ polypeptide; a truncated YopM polypeptide and truncated NIeC polypeptide; a truncated YopM polypeptide and a truncated NleB polypeptide; and a truncated IpaH9.8 polypeptide and a truncated IpaH4.5 polypeptide. In some embodiments, the constructs provided herein can exclude any one of the truncated T3SS bacterial effector polypeptides comprising a portion of the full length-bacterial effector polypeptides YopE, YopJ, YopM, NIeC, NleB, NleE, OspZ, IpaH4.5, IpaH7.8, and IpaH9.8.

Useful bacterial effector polypeptides can have the biochemical activity, target specificity and cellular effects as shown in Table 1.

TABLE 1
T3SS effector activity

| T3SS | | Biochemical | |
|---|---|---|---|
| Effector | Target | Activity | Cellular Effects |
| YopM | Caspase-1 and | LRR motif | Inhibition of I1-1beta processing, |
| | the inflammasome; | mediated binding | inflammasome activation |
| | | and sequestration | |
| YopE | Rho GTPases/Caspases | Rho GAP mimicry | Block of NF-kappaB activation |
| YopJ | MAPKs and IKB | Acetyltransferase | Block of MAPK and NF- |
| | | | kappaB signaling |
| YopP | MAPKs and IKB | Acetyltransferase | Block of MAPK and NF- |
| | | | kappaB signaling |
| IpaH4.5 | P65, TBK1 | E3 ubiquitin ligase | Inhibition of NF-kappaB |
| | | | activation and type I IFN |
| | | | activation |
| IpaH9.8 | NEMO; U2AF; GBPs | E3 ubiquitin ligase | Inhibition of U2AF - mediated |
| | | | gene expression; Inhibition of |
| | | | NF-kappaB activation; inhibition |
| | | | of GBP-mediated cell- |
| | | | autonomous immunity |
| NleC | p65 | Zinc | Cleaves and inactivates p65 |
| | | metalloprotease | |
| NleB | FADD; TRAD; RIPK | O-GlcNac | Antagonizes death receptor - |
| | | transferase | mediated apoptosis |
| OspZ | TAB2/3 | Cysteine | Block of NF-kappaB activation |
| | | methylase | and IL-8 production |

The constructs disclosed herein comprise two or more truncated T3SS bacterial effector polypeptides. A truncated bacterial effector polypeptide can be a continuous or contiguous portion of the referenced full-length polypeptide (e.g., a fragment of a polypeptide that is 10 amino acids long can be any 10 contiguous residues within that polypeptide). Thus, a truncated T3SS bacterial effector polypeptide can lie within the referenced full-length polypeptide. A truncated T3SS bacterial effector polypeptide can be at least 1, 2, 3, 4, 5, 6, 7, 10, 15, 20, 25, 30, 40, 50, 60, 70, 80, 90, 100 or more amino acid residues shorter in length than the referenced full-length polypeptide. In some embodiments, the amino acid sequence of the truncated T3SS bacterial effector polypeptide lacks 1, 2, 3, 4, 5, 6, 7, 10, 15, 20, 25, 30, 40, 50, 60, 70, 80, 90, 100 or more amino acid residues at the C-terminal relative to the referenced full-length polypeptide. In some embodiments, the amino acid sequence of the truncated T3SS bacterial effector polypeptide lacks 1, 2, 3, 4, 5, 6, 7, 10, 15, 20, 25, 30, 40, 50, 60, 70, 80, 90, 100 or more amino acid residues at the N-terminal relative to the referenced full-length polypeptide.

In some embodiments, a truncated bacterial effector polypeptide retains one or more of the activities of the referenced full-length bacterial effector polypeptide. For example, a truncated E3 ubiquitin ligase can retain all or substantially all of the E3 ubiquitin ligase activity of the referenced full-length E3 ubiquitin ligase. In some embodiments, a truncated bacterial effector polypeptide lacks or substantially lacks one or more of the activities of the referenced full-length bacterial effector polypeptide.

A referenced full-length T3SS bacterial effector polypeptide can have an amino acid sequence as set forth in SEQ ID NO. 1; SEQ ID NO. 3; SEQ ID NO. 5; SEQ ID NO. 7; SEQ ID NO. 9; SEQ ID NO. 11; SEQ ID NO. 13; SEQ ID NO. 15; or SEQ ID NO. 21. In some embodiments, a referenced full-length T3SS bacterial effector polypeptide can have an amino acid sequence at least 90% identical to an amino acid sequence as set forth in SEQ ID NO. 1; SEQ ID NO. 3; SEQ ID NO. 5; SEQ ID NO. 7; SEQ ID NO. 9; SEQ ID NO. 11; SEQ ID NO. 13; SEQ ID NO. 15; or SEQ ID NO. 21.

A construct can be a fusion protein comprising an amino acid sequence of a first truncated T3SS bacterial effector polypeptide and an amino acid sequence of a second truncated bacterial effector polypeptide. In some embodiments the amino acid sequence of the first truncated bacterial effector polypeptide and the amino acid sequence of the second truncated bacterial effector polypeptide can be contiguous, with the amino acid sequence of the first truncated bacterial effector polypeptide and the amino acid sequence of the second truncated bacterial effector polypeptide being joined by a peptide bond.

In some embodiments, the amino acid sequences of the first truncated bacterial effector polypeptide and the amino acid sequence of the second truncated bacterial effector polypeptide are connected by a linker. The linker can be a cleavable linker. Cleavable linkers can include pH sensitive linkers, for example, hydrazones; phosphoramidate-based linkers, thiomaleic acid; a proteasome specific linker, for example, Phe-Lys dipeptide linker, Val-Cit-PABC linker; an enzyme specific linker, for example, a glucuronide-MABC linker or a β-glucuronide linker; a disulfide linker, for example a: dithiocyclopeptide linker, a s Nfo-SPDB linker, or a SPDB linker; a metal assisted linker, for example a palladium linker or a iron linker; a photo-cleavable linker, for example, a nitrobenzyl linker, or a di 6-(3-succinimidyl carbonyloxymethyl-4-nitro-phenoxy)-hexanoic acid disulfide diethanol ester (SCNE) linker.

In some embodiments, a linker can include at least one amino acid residue and can be a peptide of at least or about 2, 3, 4, 5, 6, 7, 10, 15, 20, 25, 30, 40, or 50 amino acid residues. Where the linker is a single amino acid residue it can be any naturally or non-naturally occurring amino acid (e.g., Gly, Cys, Lys, Glu, or Asp) or a dipeptide including two such residues (e.g., Gly-Lys). Where the linker is a short peptide, it can be a glycine-rich peptide (which tend to be flexible) such as a peptide having the sequence [Gly-Gly-Gly-Gly-Ser]ₙ where n is an integer from 1 to 6 (SEQ ID NO: 25), inclusive or a serine-rich peptide linker. Serine rich peptide linkers include those of the formula [X-X-X-X-Gly]_{y} where up to two of the X are Thr, the remaining X are Ser, and y is an integer from 1 to 5 (SEQ ID NO: 26), inclusive (e.g., Ser-Ser-Ser-Ser-Gly (SEQ ID NO: 27), where y is greater than 1). Other linkers include rigid linkers (e.g., PAPAP (SEQ ID NO: 28) and (PT)ₙP, where n is 2, 3, 4, 5, 6, or 7 (SEQ ID NO: 29)) and α-helical linkers (e.g., A(EAAAK)ₙA, where n is 1, 2, 3, 4, or 5 (SEQ ID NO: 30)). When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the ε-amino group of Lys and the substituent. The further linker may be succinic acid, which can form an amide bond with the ε-amino group of Lys and with an amino group present in the substituent. In one embodiment, the further linker is Glu or Asp (e.g., which forms an amide bond with the ε-amino group of Lys and another amide bond with a carboxyl group present in the substituent), that is, the substituent is a NE-acylated lysine residue.

The constructs disclosed herein can further comprise a protein transduction domain (PTD), that is, an amino acid sequence that mediates translocation across the cell membrane. Useful protein transduction domains include a YopM protein transduction domain and an IpaH protein transduction domain. An exemplary YopM protein transduction domain can have an amino acid sequence set forth in SEQ ID NO. 17. An exemplary IpaH9.8 transduction domain can have an amino acid sequence as set forth in amino acids 1-57 of SEQ ID NO. 11. The protein transduction domain and the construct comprising a first truncated T3SS bacterial effector polypeptide sequence and second truncated T3SS bacterial effector polypeptide sequence can be a fusion protein. In some embodiments, the amino acid sequence of the protein transduction domain and the first truncated bacterial effector polypeptide and the amino acid sequence of the second truncated bacterial effector polypeptide can be contiguous, with the amino acid sequence of the protein transduction domain and the first truncated bacterial effector polypeptide and the amino acid sequence of the second truncated bacterial effector polypeptide being joined by peptide bonds. In some embodiments, the protein transduction domain and the amino acid sequence of the first truncated bacterial effector polypeptide and the amino acid sequence of the second truncated bacterial effector polypeptide are connected by a linker, that is, any of the linkers described above.

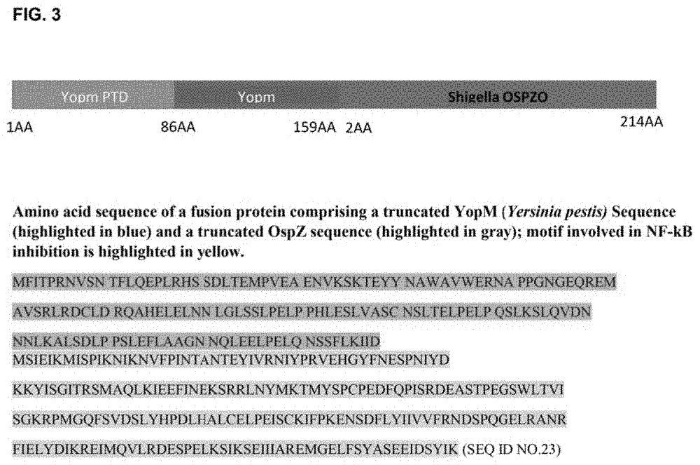

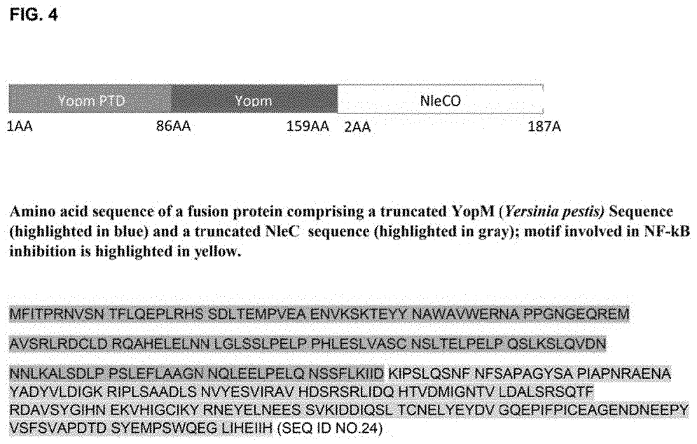

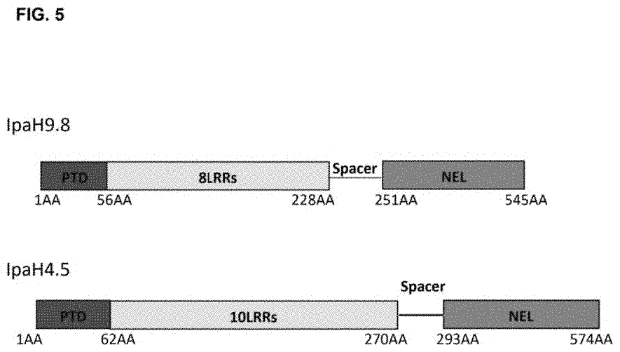

Exemplary constructs and the amino acid sequences of such constructs are shown in FIGS. 3 and 4. FIG. 3 depicts a fusion protein comprising a YopM protein transduction domain, a truncated YopM polypeptide, and a truncated OspZ polypeptide. The amino acid sequence of the fusion protein shown in FIG. 3 is SEQ ID No.: 23. FIG. 4 depicts a fusion protein comprising a YopM protein transduction domain, a truncated YopM polypeptide, and a truncated NIeC polypeptide. The amino acid sequence of the fusion protein shown in FIG. 4 is SEQ ID No.: 24.

The polypeptides provided herein can have one or more amino acid additions, subtractions, or substitutions relative to a native polypeptide amino acid sequence (also referred to herein as “variant” T3SS polypeptides) can be prepared and modified as described herein. In some cases, amino acid substitutions can be made by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of useful conservative substitutions can include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

In some embodiments, a polypeptide can include one or more non-conservative substitutions. Non-conservative substitutions typically entail exchanging a member of one of the classes described above for a member of another class. Such production can be desirable to provide large quantities or alternative embodiments of such constructs. Whether an amino acid change results in a functional polypeptide can readily be determined by assaying the specific activity of the peptide variant.

A polypeptide provide herein can be obtained by expression of a recombinant nucleic acid encoding the polypeptide or by chemical synthesis. For example, recombinant technology using expression vectors encoding a polypeptide provide herein can be used. The resulting polypeptides then can be purified using, for example, affinity chromatographic techniques and HPLC. The extent of purification can be measured by any appropriate method, including but not limited to: column chromatography, polyacrylamide gel electrophoresis, or high-performance liquid chromatography. A polypeptide provide herein can be designed or engineered to contain a tag sequence that allows the polypeptide to be purified (e.g., captured onto an affinity matrix). For example, a tag such as c-myc, hemagglutinin, polyhistidine, or Flag™ tag (Kodak) can be used to aid polypeptide purification. Such tags can be inserted anywhere within the polypeptide including at either the carboxyl or amino termini.

The polypeptides disclosed herein can be isolated from inside or outside of the host cell or the medium in which the cell has been cultured and purified as substantially pure and homogenous polypeptides. A substantially pure polypeptide can be for example, a polypeptide that is removed from its the host cell or medium in which it was produced and can be at least 60%, at least 70%, at least 80%, or at least 90% pure, that is free or substantially free from other components such as unrelated polypeptides, lipids, nucleic acids, or carbohydrates. Polypeptides may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography. Chromatography can be carried out using liquid phase chromatography such as HPLC and FPLC.

A polypeptide provided herein can be formulated as a pharmaceutical composition by admixture with pharmaceutically acceptable non-toxic excipients or carriers. Such compositions can be administered to a subject in need thereof in an amount effective to treat an inflammatory condition. Pharmaceutical compositions may be prepared for oral or parenteral administration, for example, nasal, sublingual, buccal, intra-arterial, intra-articular, intra-cardiac, intradermal, intramuscular, intraocular, intra-osseous, intraperitoneal, intrathecal, intravenous, intravesicular, intravitreal, subcutaneous, transdermal, perivascular, intracerebral, transmucosal administration. Intra-articular administration may be useful for treatment of inflammatory conditions of the joints. Compositions formulated for parenteral administration can be in the form of liquid solutions or suspensions in aqueous physiological buffer solutions; for oral administration, particularly in the form of tablets or capsules; or for intranasal administration, particularly in the form of powders, nasal drops, or aerosols.

The excipient or carrier can vary depending upon the formulation and the route of administration. Pharmaceutical carriers are described in Remington's Pharmaceutical Sciences (E. W. Martin) and in the USP/NF (United States Pharmacopeia and the National Formulary). Exemplary excipients can include sugars, for example, lactose, dextrose, sucrose, sorbitol, mannitol; starches, gum acacia, calcium phosphate, alginates, tragacanth, gelatin, calcium silicate, microcrystalline cellulose, polyvinylpyrrolidone, cellulose, water, syrup, and methyl cellulose. In some embodiments, the formulations can include a lubricating agent, a wetting agent, an emulsifying agent, a preservative, a sweetener, or a flavoring.

Formulations for parenteral administration may contain as common excipients sterile water or saline, polyalkylene glycols such as polyethylene glycol, oils of vegetable origin, hydrogenated naphthalenes, and the like. In particular, biocompatible, biodegradable lactide polymer, lactide/glycolide copolymer, or polyoxethylene-polyoxypropylene copolymers are examples of excipients for controlling the release of the polypeptide in vivo. Other suitable parenteral delivery systems include ethylene-vinyl acetate copolymer particles, osmotic pumps, implantable infusion systems, and liposomes. Formulations for inhalation administration may contain excipients such as lactose, if desired. Inhalation formulations may be aqueous solutions containing, for example, polyoxyethylene-9-lauryl ether, glycocholate and deoxycholate, or they may be oily solutions for administration in the form of nasal drops. If desired, the compounds can be formulated as gels to be applied intranasally. Formulations for parenteral administration may also include glycocholate for buccal administration

For oral administration, tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g., potato starch or sodium starch glycolate); or wetting agents (e.g., sodium lauryl sulfate). Tablets can be coated by methods known in the art. Preparations for oral administration can also be formulated to give controlled release of the compound.

Nasal preparations can be presented in a liquid form or as a dry product. Nebulized aqueous suspensions or solutions can include carriers or excipients to adjust pH and/or tonicity.

In some embodiments, the pharmaceutical compositions can be formulated to modulate the release of the active ingredient. The pharmaceutical compositions can also be formulated so as to provide quick, sustained or delayed release of the active ingredient after administration to the patient. A polypeptide provided herein can be formulated as a sustained release dosage form. For example, a polypeptide can be formulated into a controlled release formulation. In some embodiments, coatings, envelopes, or protective matrices can be formulated to contain one or more of the polypeptides provided herein. In some embodiments, such coatings, envelopes, and protective matrices can be used to coat indwelling devices such as stents, catheters, and peritoneal dialysis tubing. In some cases, a polypeptide provided herein can be incorporated into a polymeric substances, liposomes, microemulsions, microparticles, nanoparticles, or waxes.

Also provided are nucleic acids encoding any of the constructs disclosed herein. An isolated nucleic acid refers to a nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

Isolated nucleic acids also include any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid (e.g., a nucleic acid encoding a polypeptide comprising or consisting of the amino acid sequence set forth in SEQ ID NO. 23 and SEQ ID NO. 24) can be made using molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be RNA and DNA, including mRNA, cDNA, genomic DNA, synthetic (e.g., chemically synthesized) DNA, and nucleic acid analogs. The nucleic acid can be double-stranded or single-stranded, and where single-stranded, can be the sense strand or the antisense strand. In addition, nucleic acid can be circular or linear. Nucleic acid analogs can be modified at the base moiety, sugar moiety, or phosphate backbone to improve, for example, stability, hybridization, or solubility of a nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone and the four bases are retained. In addition, the deoxyphosphate backbone can be replaced with, for example, a phosphorothioate or phosphorodithioate backbone, a phosphoroamidite, or an alkyl phosphotriester backbone.

A nucleic acid provided herein can comprise or consist of any of the nucleic acid sequences set forth in sequence set forth in SEQ ID NO. 2; SEQ ID NO. 4; SEQ ID NO. 6; SEQ ID NO. 8; SEQ ID NO. 10; SEQ ID NO. 12; SEQ ID NO. 14; SEQ ID NO. 16; SEQ ID NO. 18, SEQ ID NO. 20, or SEQ ID NO. 22. In some embodiments, the nucleic acid can comprise a truncated nucleic acid of any of SEQ ID NO. 2; SEQ ID NO. 4; SEQ ID NO. 6; SEQ ID NO. 8; SEQ ID NO. 10; SEQ ID NO. 12; SEQ ID NO. 14; SEQ ID NO. 16; SEQ ID NO. 18, SEQ ID NO. 20, or SEQ ID NO. 22.

The nucleic acids that encode a first truncated T3SS bacterial effector polypeptide sequence and second truncated T3SS bacterial effector polypeptide sequence include those that are codon optimized. For expression, the nucleic acids can be incorporated into a vector (e.g., a plasmid or viral vector), and such vectors are encompassed by the invention. The nucleic acids can be operably linked to a regulatory region suitable for use in either a prokaryotic or a eukaryotic system. In specific embodiments, the regulatory region can be, for example, a promoter or enhancer. Useful promoters include cell type-specific promoters, tissue-specific promoters, constitutively active promoters, and broadly expressing promoters. Host cells including vectors that express a polypeptide of the invention are also encompassed by the invention, and these cells can be prokaryotic (e.g., bacterial) or eukaryotic (e.g., mammalian).

Typically, an isolated nucleic acid provided herein is at least 10 nucleotides in length (e.g., 10, 15, 20, 25, 30, 35, 40, 50, 75, 100, 200, 300, 350, 400, or more nucleotides in length). Nucleic acid molecules that are less than full-length can be useful, for example, as primers or probes. Isolated nucleic acid molecules can be produced molecular cloning and chemical nucleic acid synthesis techniques. For example, polymerase chain reaction (PCR) techniques can be used. Isolated nucleic acids also can be chemically synthesized, either as a single nucleic acid molecule (e.g., using automated DNA synthesis in the 3′ to 5′ direction using phosphoramidite technology) or as a series of oligonucleotides, which then can be ligated into a vector.

Also provided are methods of treating a subject having or at risk for an inflammatory condition by administering a therapeutically effective amount of a pharmaceutical composition comprising any of the constructs disclosed herein. In some embodiments, the subject (e.g., a human patient) in need of the treatment is diagnosed with, suspected of having, or at risk for an inflammatory condition. Exemplary inflammatory conditions include but are not limited to, inflammatory conditions found in arthritis and joint diseases, for example, rheumatoid arthritis and osteoarthritis; cardiovascular diseases; allergies; asthma; chronic obstructive pulmonary disease; diabetes; gastrointestinal diseases, for example, inflammatory bowel disease, Crohn's disease, and iliocolitis; cancer, for example, kidney cancer, prostate cancer, ovarian cancer, hepatocellular cancer, pancreatic cancer, colorectal cancer, lung cancer, and mesothelioma; chronic kidney disease; and Alzheimer's disease.

In general, a treatment can include one or more of inhibiting the inflammatory condition in an individual who is experiencing or displaying the pathology or symptomatology of the disease, condition or disorder (i.e., arresting further development of the pathology and/or symptomatology). A treatment can also include ameliorating the inflammatory condition in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

A subject can be a human or a nonhuman animal. Exemplary non-human species include, without limitation, nonhuman primates; domestic animals, for example horses, pigs, cows, sheep; cats, dogs, mice or rats. Subjects suitable for treatment may be identified by the detection of symptoms commonly associated with inflammatory conditions, such as pain, fatigue, gastrointestinal symptoms such as constipation, diarrhea, and acid reflux, weight gain, and frequent infections. Subjects suitable for treatment can also be identified by laboratory tests, including for example, serum protein electrophoresis (SPE), high-sensitivity C-reactive protein, fibrinogen, and detection of pro-inflammatory cytokines.

A therapeutically effective amount can be the amount of active compound or pharmaceutical agent that elicits the biological or medicinal response that is being sought in a tissue, system, animal, individual or human by a researcher, veterinarian, medical doctor or other clinician.

The compositions provided herein can be administered in combination with one or more conventional therapeutic agents, including treatments for arthritis and joint diseases, for example, rheumatoid arthritis and osteoarthritis; cardiovascular diseases; allergies; asthma; chronic obstructive pulmonary disease; diabetes; gastrointestinal diseases, for example, inflammatory bowel disease, Crohn's disease, and iliocolitis; cancer, for example, kidney cancer, prostate cancer, ovarian cancer, hepatocellular cancer, pancreatic cancer, colorectal cancer, lung cancer, and mesothelioma; chronic kidney disease; and Alzheimer's disease.

In general, the invention features constructs that can include two or more truncated T3SS bacterial effector polypeptides comprising a portion of the full length-bacterial effector polypeptides. In one aspect, a construct can include a truncated YopM polypeptide linked to a truncated T3SS cysteine methyltransferase polypeptide. The truncated YopM polypeptide can have an amino acid sequence at least 90% identical to the amino acid sequence as set forth in SEQ ID NO.: 19. The truncated YopM polypeptide can have the amino acid sequence set forth in SEQ ID NO.: 19. The truncated T3SS cysteine methyltransferase polypeptide can include a portion of an OspZ polypeptide having an amino acid sequence as set forth in SEQ ID NO.: 3. The truncated OspZ polypeptide can have an amino acid sequence at least 90% identical amino acid 226-446 of SEQ ID NO.: 3. The truncated OspZ polypeptide can have the amino acid sequence as set forth in amino acids 226-446 of SEQ ID NO.: 3. The construct can further include a protein transduction domain, for example, a YopM protein transduction domain as set forth in SEQ ID NO.: 17. In some embodiments, the construct comprises an amino acid sequence as set forth in SEQ ID NO.: 23.

In another aspect, a construct can include a truncated YopM polypeptide linked to a truncated T3SS zinc metalloprotease polypeptide. The truncated YopM polypeptide can have an amino acid sequence at least 90% identical to the amino acid sequence as set forth in SEQ ID NO.: 19. The truncated YopM polypeptide can have the amino acid sequence set forth in SEQ ID NO.: 19. The truncated T3SS zinc metalloprotease polypeptide can include a portion of an NIeC polypeptide having an amino acid sequence as set forth in SEQ ID NO.: 5. The truncated NIeC polypeptide can have an amino acid sequence at least 90% identical amino acids 2-187 of SEQ ID NO.: 5. In some embodiments, the truncated NIeC polypeptide can have the amino acid sequence as set forth in amino acids 2-187 of SEQ ID NO.: 5. In some embodiments, the truncated NIeC polypeptide has the amino acid sequence as set forth in amino acids 2-187 of SEQ ID NO.: 5. The construct can further include a protein transduction domain, for example, a YopM protein transduction domain as set forth in SEQ ID NO.: 17. In some embodiments, the construct has an amino acid sequence as set forth in SEQ ID NO.: 24.

In one aspect, a construct can include a truncated YopM polypeptide linked to a truncated T3SS O-GlcNac transferase. The truncated YopM polypeptide can have an amino acid sequence at least 90% identical to the amino acid sequence as set forth in SEQ ID NO.: 19. The truncated YopM polypeptide can have the amino acid sequence set forth in SEQ ID NO.: 19. The truncated T3SS O-GlcNac transferase can include a portion of an NleB polypeptide having an amino acid sequence as set forth in SEQ ID NO.: 9. The truncated NleB polypeptide can have an amino acid sequence at least 90% identical amino acids 2-226 of SEQ ID NO.: 9. The truncated NleB polypeptide can have an amino acid sequence as set forth in amino acids 2-226 of SEQ ID NO.: 9. The construct can further include a protein transduction domain, for example, a YopM protein transduction domain as set forth in SEQ ID NO.: 17.

In one aspect, a construct can include a truncated first T3SS E3 ubiquitin ligase polypeptide linked to a truncated second T3SS E3 ubiquitin ligase. The first and second truncated T3SS E3 ubiquitin ligase polypeptides can be different. The truncated first E3 ubiquitin ligase can include a portion of an IpaH9.8 polypeptide having an amino acid sequence as set forth in SEQ ID NO.: 11. The truncated first IpaH9.8 polypeptide comprises an amino acid sequence at least 90% identical amino acid 56-228 of SEQ ID NO.: 11. The truncated second E3 ubiquitin ligase comprises a portion of an IpaH4.5 polypeptide having an amino acid sequence as set forth in SEQ ID NO.: 13. The truncated second IpaH4.5 polypeptide comprises an amino acid sequence at least 90% identical amino acid 62-270 of SEQ ID NO.: 13. The construct can further include a protein transduction domain, for example, an IpaH9.8 protein transduction domain as set forth in amino acids 1-57 of SEQ ID NO. 11.

In one aspect, a construct can include a RhoGTPase modulator linked to a cysteine methyltransferase, wherein the RhoGTPase modulator is linked to the cysteine methyltransferase by a pH sensitive linker. The RhoGTPase modulator can be a YopE polypeptide having an amino acid sequence as set forth in SEQ ID NO.: 1. The cysteine methyltransferase can be an OspZ polypeptide having an amino acid sequence as set forth in SEQ ID NO.: 5. The pH sensitive linker comprises a hydrazine, a phosphoramidate-based linker, or thiomaleic acid.

In one aspect, a construct can include a truncated YopM polypeptide linked to an acetyltransferase. The truncated YopM polypeptide can have an amino acid sequence at least 90% identical to the amino acid sequence as set forth in SEQ ID NO.: 19. The truncated YopM polypeptide can have the amino acid sequence set forth in SEQ ID NO.: 19. The acetyltransferase can be a YopJ polypeptide having an amino acid sequence as set forth in SEQ ID NO.: 9 comprising a mutation at cysteine 172.

In one aspect, a construct can include a truncated YopE polypeptide linked to an acetyltransferase. The truncated YopE polypeptide can include a portion of YopE polypeptide having an amino acid sequence as set forth in SEQ ID NO.: 1. The construct can further include a protein transduction domain, for example, an IpaH9.8 protein transduction domain as set forth in amino acids 1-57 of SEQ ID NO. 11.

In one aspect, construct can further comprise a protein transduction domain. The protein transduction domain can be a YopM protein transduction domain. The YopM protein transduction domain can have an amino acid sequence as set forth in SEQ ID NO.: 19. The protein transduction domain can be an IpaH9.8 protein transduction domain. The IpaH9.8 protein transduction domain can have an amino acid sequence as set forth in amino acids 1-56 of SEQ ID NO.: 23.

In one aspect, any of the constructs can comprise a fusion protein. The first truncated T3SS bacterial effector polypeptide and the second truncated T3SS bacterial effector polypeptide can be joined by a linker. The linker can be cleavable linker. The cleavable linker can be a pH sensitive linker. The pH sensitive linker can be selected from the group consisting of a hydrazine, a phosphoramidate-based linker, and a thiomaleic acid.

In one aspect, also provided are nucleic acids encoding any of the constructs disclosed herein.

In one aspect, the construct can be formulated as a pharmaceutical composition comprising the construct and a pharmaceutically acceptable carrier.

In one aspect, the present application features a method of treating a subject having or at risk for an inflammatory condition, the method including administering to the subject a therapeutically effective amount of a pharmaceutical composition including a construct that can include a first truncated T3SS bacterial effector polypeptide and the second truncated T3SS bacterial effector polypeptide and a pharmaceutically acceptable carrier. The method can include the step of identifying a subject. The inflammatory condition can be a gastrointestinal disorder, a musculoskeletal disorder, autoimmune disorder, or a skin disorder. In one aspect, the inflammatory condition can include inflammatory bowel disease, Crohn's disease, rheumatoid arthritis, osteoarthritis, cancer, allergies, cardiovascular disease, chronic obstructive pulmonary disease, and diabetes.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

We analyzed the effect of the E3 ubiquitin ligases IpaH7.8 and IpaH9.8 on cytokine release from THP-1 cells. THP-1 cells were cultured in the presence and absence of increasing amounts (0.25 μg, 0.5 μg and 1.0 μg) of recombinant IpaH7.8 or IpaH9.8. For IpaH7.8, 0.25 μg of protein is approximately 3.87 nM. For IpaH9.8, 0.25 μg of protein is approximately 4.0 nM. Cultures were then treated with lipopolysaccharide (LPS) to induce cytokine release.

As shown in Table 2, recombinant IpaH7.8 inhibited the release of IL-1β, TNF-α, MCP-1, IL-6, IL-8, IL-23. As shown in Table 3, recombinant IpaH9.8 produced a dose-dependent inhibition of release of IL-1β, TNF-α, and MCP-1, IL-6, IL-8, IL-23. These data showed that low nanomolar concentrations of the E3 ubiquitin ligases IpaH7.8 and IpaH9.8 could effectively down regulate cytokine levels in THP-1 cells.

TABLE 2
Effect of IpaH7.8 on cytokine release

| Released | Treatment | | | | |
|---|---|---|---|---|---|
| cytokine | | | LPS + | LPS + | LPS + |
| concentration | No | LPS | 0.25 μg | 0.5 μg | 1.0 μg |
| (pg/ml) | additions | only | IpaH7.8 | IpaH7.8 | IpaH7.8 |
| IL-1β | 3.02 | 70.50 | 11.69 | 8.42 | 4.44 |
| TNF-α | 2 | 279.56 | 130.26 | 113.94 | 198.3 |
| MCP-1 | 6.60 | 12204.78 | 411.6 | 33.73 | 22.11 |
| IL-6 | 2 | 148.28 | 38.74 | 55.15 | 2 |
| IL-8 | 2 | 1320.88 | 319.22 | 314.55 | 60 |
| IL-23 | 4 | 1320.88 | 319.22 | 314.55 | 60 |

TABLE 3
Effect of IpaH9.8 on cytokine release

| Released | Treatment | | | | |
|---|---|---|---|---|---|
| cytokine | | | LPS + | LPS + | LPS + |
| concentration | No | LPS | 0.25 μg | 0.5 μg | 1.0 μg |
| (pg/ml) | additions | only | IpaH9.8 | IpaH9.8 | IpaH9.8 |
| IL-1β | 3.02 | 70.50 | 14.82 | 9.94 | 4.01 |
| TNF-α | 2 | 279.56 | 158.16 | 93.21 | 66.97 |
| MCP-1 | 6.60 | 12204.78 | 7409.46 | 633.82 | 395.29 |
| IL-6 | 2 | 148.28 | 81.52 | 54.04 | 41.3 |
| IL-8 | 4 | 1320.88 | 264.15 | 228.84 | 161.25 |
| IL-23 | 4 | 1320.88 | 264.15 | 161.25 | 126.83 |

The invention is further described by the following numbered paragraphs:

1. A construct comprising two or more truncated T3SS bacterial effector polypeptides, wherein each truncated T3SS bacterial effector polypeptide comprises a portion of the corresponding full length T3SS bacterial effector polypeptide.
2. The construct of paragraph 1, wherein the truncated T3SS bacterial effector polypeptides retain one or more activities of the corresponding full length-T3SS bacterial effector polypeptides.
3. The construct of paragraph 1 wherein the T3SS bacterial effector polypeptides are selected from the group consisting of an E3 ubiquitin ligase, a RhoGTPase modulator, a cysteine methyltransferase, a zinc metalloprotease, an acetyltransferase, an O-GlcNac transferase, and an LRR motif binding and sequestration polypeptide.
4. The construct of paragraph 3 wherein the E3 ubiquitin ligase is IpaH 7.8 or IpaH9.8; the RhoGTPase modulator is YopE; the cysteine methyltransferase is OspZ or NleE); the zinc metalloprotease is NIeC; the acetyltransferase is YopJ; the O-GlcNac transferase is NleB; and the LRR motif binding and sequestration polypeptide is YopM.
5. The construct of paragraph 1, wherein the construct comprises a truncated YopM polypeptide linked to a truncated T3SS cysteine methyltransferase polypeptide.
6. The construct of paragraph 5, wherein the truncated T3SS cysteine methyltransferase polypeptide comprises a portion of an OspZ polypeptide having an amino acid sequence as set forth in SEQ ID NO.: 3.
7. The construct of paragraph 6, wherein the truncated OspZ polypeptide comprises an amino acid sequence at least 90% identical to amino acids 226-446 of SEQ ID NO.: 3.
8. The construct of paragraph 7, wherein the truncated OspZ polypeptide has the amino acid sequence as set forth in amino acids 226-446 of SEQ ID NO.: 3.
9. The construct of paragraph 5, wherein the construct comprises an amino acid sequence as set forth in SEQ ID NO.: 23.
10. The construct of paragraph 1, wherein the construct comprises a truncated YopM polypeptide linked to a truncated T3SS zinc metalloprotease polypeptide.
11. The construct of paragraph 10, wherein the truncated T3SS zinc metalloprotease polypeptide comprises a portion of an NIeC polypeptide having an amino acid sequence as set forth in SEQ ID NO.: 5.
12. The construct of paragraph 11, wherein the truncated NIeC polypeptide comprises an amino acid sequence at least 90% identical to amino acids 2-187 of SEQ ID NO.: 5.
13. The construct of paragraph 12, wherein the truncated NIeC polypeptide has the amino acid sequence as set forth in amino acids 2-187 of SEQ ID NO.: 5.
14. The construct of paragraph 10, wherein the construct comprises an amino acid sequence as set forth in SEQ ID NO.: 24.
15. The construct of paragraph 1, wherein the construct comprises a truncated YopM polypeptide linked to a truncated T3SS O-GlcNac transferase.
16. The construct of paragraph 15, wherein the truncated T3SS O-GlcNac transferase comprises a portion of an NleB polypeptide having an amino acid sequence as set forth in SEQ ID NO.: 9.
17. The construct of paragraph 16, wherein the truncated NleB polypeptide comprises an amino acid sequence at least 90% identical to amino acids 2-226 of SEQ ID NO.: 9.
18. The construct of paragraph 17, wherein the truncated NleB polypeptide has an amino acid sequence as set forth in amino acids 2-226 of SEQ ID NO.: 9.
19. The construct of paragraph 1, wherein the construct comprises a truncated first T3SS E3 ubiquitin ligase polypeptide linked to a truncated second T3SS E3 ubiquitin ligase.
20. The construct of paragraph 19, where in the first and second truncated T3SS E3 ubiquitin ligase polypeptides are different.
21. The construct of paragraph 19, wherein the truncated first E3 ubiquitin ligase comprises a portion of an IpaH9.8 polypeptide having an amino acid sequence as set forth in SEQ ID NO.: 11.
22. The construct of paragraph 20, wherein the truncated first IpaH9.8 polypeptide comprises an amino acid sequence at least 90% identical to amino acid 56-228 of SEQ ID NO.: 11.
23. The construct of paragraph 22, wherein the truncated second E3 ubiquitin ligase comprises a portion of an IpaH4.5 polypeptide having an amino acid sequence as set forth in SEQ ID NO.: 13.
24. The construct of paragraph 23, wherein the truncated second IpaH4.5 polypeptide comprises an amino acid sequence at least 90% identical to amino acid 62-270 of SEQ ID NO.: 13.
25. The construct of paragraph 1, wherein the construct comprises a RhoGTPase modulator linked to a cysteine methyltransferase.
26. The construct of paragraph 25, wherein the RhoGTPase modulator is a YopE polypeptide having an amino acid sequence as set forth in SEQ ID NO.: 1.
27. The construct of paragraph 26, wherein the cysteine methyltransferase is an OspZ polypeptide having an amino acid sequence as set forth in SEQ ID NO.: 5.
28. The construct of paragraph 1, wherein the construct comprises a truncated YopM polypeptide linked to a truncated acetyltransferase polypeptide.
29. The construct of paragraph 28, wherein the truncated acetyltransferase comprises a portion of a YopJ polypeptide having an amino acid sequence as set forth in SEQ ID NO.: 9.
30. The construct of paragraph 28, wherein the truncated YopJ polypeptide comprises a point mutation at cysteine 172 of SEQ ID NO.: 9.
31. The construct of any one of paragraph 1-18, wherein the truncated YopM polypeptide has an amino acid sequence at least 90% identical to the amino acid sequence as set forth in SEQ ID NO.: 19.
32. The construct of paragraph 31, wherein the truncated YopM polypeptide has the amino acid sequence set forth in SEQ ID NO.: 19.
33. The construct of any one of paragraphs 1-32, further comprising a protein transduction domain.
34. The construct of paragraph 27, wherein the protein transduction domain is a YopM protein transduction domain.
35. The construct of paragraph 34, where in the YopM protein transduction domain has an amino acid sequence as set forth in SEQ ID NO.: 17.
36. The construct of paragraph 27, where in the protein transduction domain is an IpaH9.8 protein transduction domain.
37. The construct of paragraph 27, where in the IpaH9.8 protein transduction domain has an amino acid sequence as set forth in amino acids 2-56 of SEQ ID NO.: 11.
38. The construct of any one of paragraphs 1-37, wherein the construct comprises a fusion protein.
39. The construct of any one of paragraphs 1-38, wherein the two or more truncated T3SS bacterial effector polypeptides are joined by a linker.
40. The construct of paragraph 39, where in the linker is a cleavable linker.
41. The construct of paragraph 40, wherein the cleavable linker is a pH sensitive linker.
42. The construct of paragraph 41, wherein the pH sensitive linker comprises a hydrazine, a phosphoramidate-based linker, or thiomaleic acid.
43. The construct of paragraph 39, wherein the linker is a covalent bond.
44. A nucleic acid encoding any one of the constructs of paragraphs 1-43.
45. A pharmaceutical composition comprising the construct of any one of paragraphs 1-44 and a pharmaceutically acceptable carrier.
46. A method of treating a subject having an inflammatory disorder, the method comprising administering a therapeutically effective amount of the pharmaceutical composition of paragraph 45.
47. The method of paragraph 46, wherein the inflammatory disorder is a skin disorder, a gastrointestinal disorder, or a musculoskeletal disorder.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A construct comprising two or more truncated T3SS bacterial effector polypeptides, wherein each truncated T3SS bacterial effector polypeptide comprises a portion of the corresponding full length T3SS bacterial effector polypeptide.

2. The construct of claim 1, wherein the truncated T3SS bacterial effector polypeptides retain one or more activities of the corresponding full length-T3SS bacterial effector polypeptides.

3. The construct of claim 1 wherein the T3SS bacterial effector polypeptides are selected from the group consisting of an E3 ubiquitin ligase, a RhoGTPase modulator, a cysteine methyltransferase, a zinc metalloprotease, an acetyltransferase, an O-GlcNac transferase, and an LRR motif binding and sequestration polypeptide.

4. The construct of claim 3 wherein the E3 ubiquitin ligase is IpaH 7.8 or IpaH9.8; the RhoGTPase modulator is YopE; the cysteine methyltransferase is OspZ or NleE); the zinc metalloprotease is NIeC; the acetyltransferase is YopJ; the O-GlcNac transferase is NleB; and the LRR motif binding and sequestration polypeptide is YopM.

5. The construct of claim 1, wherein the construct comprises a truncated YopM polypeptide linked to a truncated T3SS cysteine methyltransferase polypeptide.

6. The construct of claim 5, wherein the truncated T3SS cysteine methyltransferase polypeptide comprises a portion of an OspZ polypeptide having an amino acid sequence as set forth in SEQ ID NO.: 3.

7. The construct of claim 6, wherein the truncated OspZ polypeptide comprises an amino acid sequence at least 90% identical to amino acids 226-446 of SEQ ID NO.: 3.

8. The construct of claim 7, wherein the truncated OspZ polypeptide has the amino acid sequence as set forth in amino acids 226-446 of SEQ ID NO.: 3.

9. The construct of claim 5, wherein the construct comprises an amino acid sequence as set forth in SEQ ID NO.: 23.

10. The construct of claim 1, wherein the construct comprises a truncated YopM polypeptide linked to a truncated T3SS zinc metalloprotease polypeptide.

11. The construct of claim 10, wherein the truncated T3SS zinc metalloprotease polypeptide comprises a portion of an NIeC polypeptide having an amino acid sequence as set forth in SEQ ID NO.: 5.

12. The construct of claim 11, wherein the truncated NIeC polypeptide comprises an amino acid sequence at least 90% identical to amino acids 2-187 of SEQ ID NO.: 5.

13. The construct of claim 12, wherein the truncated NIeC polypeptide has the amino acid sequence as set forth in amino acids 2-187 of SEQ ID NO.: 5.

14. The construct of claim 10, wherein the construct comprises an amino acid sequence as set forth in SEQ ID NO.: 24.

15. The construct of claim 1, wherein the construct comprises a truncated YopM polypeptide linked to a truncated T3SS O-GlcNac transferase.

16. The construct of claim 15, wherein the truncated T3SS O-GlcNac transferase comprises a portion of an NleB polypeptide having an amino acid sequence as set forth in SEQ ID NO.: 9.

17. The construct of claim 16, wherein the truncated NleB polypeptide comprises an amino acid sequence at least 90% identical to amino acids 2-226 of SEQ ID NO.: 9.

18. The construct of claim 17, wherein the truncated NleB polypeptide has an amino acid sequence as set forth in amino acids 2-226 of SEQ ID NO.: 9.

19. The construct of claim 1, wherein the construct comprises a truncated first T3SS E3 ubiquitin ligase polypeptide linked to a truncated second T3SS E3 ubiquitin ligase.

20. The construct of claim 19, where in the first and second truncated T3SS E3 ubiquitin ligase polypeptides are different.

21. The construct of claim 19, wherein the truncated first E3 ubiquitin ligase comprises a portion of an IpaH9.8 polypeptide having an amino acid sequence as set forth in SEQ ID NO.: 11.

22. The construct of claim 20, wherein the truncated first IpaH9.8 polypeptide comprises an amino acid sequence at least 90% identical to amino acid 56-228 of SEQ ID NO.: 11.

23. The construct of claim 22, wherein the truncated second E3 ubiquitin ligase comprises a portion of an IpaH4.5 polypeptide having an amino acid sequence as set forth in SEQ ID NO.: 13.

24. The construct of claim 23, wherein the truncated second IpaH4.5 polypeptide comprises an amino acid sequence at least 90% identical to amino acid 62-270 of SEQ ID NO.: 13.

25. The construct of claim 1, wherein the construct comprises a RhoGTPase modulator linked to a cysteine methyltransferase.

26. The construct of claim 25, wherein the RhoGTPase modulator is a YopE polypeptide having an amino acid sequence as set forth in SEQ ID NO.: 1.

27. The construct of claim 26, wherein the cysteine methyltransferase is an OspZ polypeptide having an amino acid sequence as set forth in SEQ ID NO.: 5.

28. The construct of claim 1, wherein the construct comprises a truncated YopM polypeptide linked to a truncated acetyltransferase polypeptide.

29. The construct of claim 28, wherein the truncated acetyltransferase comprises a portion of a YopJ polypeptide having an amino acid sequence as set forth in SEQ ID NO.: 9.

30. The construct of claim 28, wherein the truncated YopJ polypeptide comprises a point mutation at cysteine 172 of SEQ ID NO.: 9.

31. The construct of claim 1, wherein the truncated YopM polypeptide has an amino acid sequence at least 90% identical to the amino acid sequence as set forth in SEQ ID NO.: 19.

32. The construct of claim 31, wherein the truncated YopM polypeptide has the amino acid sequence set forth in SEQ ID NO.: 19.

33. The construct of claim 1, further comprising a protein transduction domain.

34. The construct of claim 27, wherein the protein transduction domain is a YopM protein transduction domain.

35. The construct of claim 34, where in the YopM protein transduction domain has an amino acid sequence as set forth in SEQ ID NO.: 17.

36. The construct of claim 27, where in the protein transduction domain is an IpaH9.8 protein transduction domain.

37. The construct of claim 27, where in the IpaH9.8 protein transduction domain has an amino acid sequence as set forth in amino acids 2-56 of SEQ ID NO.: 11.

38. The construct of claim 1, wherein the construct comprises a fusion protein.

39. The construct of claim 1, wherein the two or more truncated T3SS bacterial effector polypeptides are joined by a linker.

40. The construct of claim 39, where in the linker is a cleavable linker.

41. The construct of claim 40, wherein the cleavable linker is a pH sensitive linker.

42. The construct of claim 41, wherein the pH sensitive linker comprises a hydrazine, a phosphoramidate-based linker, or thiomaleic acid.

43. The construct of claim 39, wherein the linker is a covalent bond.

44. A nucleic acid encoding the constructs of claim 1.

45. A pharmaceutical composition comprising the construct of claim 1 and a pharmaceutically acceptable carrier.

46. A method of treating a subject having an inflammatory disorder, the method comprising administering a therapeutically effective amount of the pharmaceutical composition of claim 45.

47. The method of claim 46, wherein the inflammatory disorder is a skin disorder, a gastrointestinal disorder, or a musculoskeletal disorder.


SEQUENCE LISTING (30 sequences)

SEQ ID NO: 1 - protein, 219 aa, Yersinia pestis
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATGSGPLRGSITQCQGLMQFCGGELQAEASAILNTPVCGIPFSQWGTVGGAASAYVASGVDLTQAANEIKGLGQQMQQLLSLM

SEQ ID NO: 2 - DNA, 1152 bp, Yersinia pestis
gaattccccaactttgacaccgataaccggttcaatagtatctggaatagacagcgaaagttgttgaaataattgagtgatagcttgttcaaatgaatacatttgatctcctaatagttagataaaatatcaacttaaccaaagcactctcggcagaccatcaattttagcctataatttttagtttttattttgtctaatataacaacaaaaacagcagcggttttttatataaccaccggctattttcccactaagataaccttgttttaatagccaagggaataaatagtcatgaaaatatcatcatttatttctacatcactgcccctgccggcatcagtgtcaggatctagcagcgtaggagaaatgtctgggcgctcagtctcacagcaaaaaagtgatcaatatgcaaacaatctggccgggcgcactgaaagccctcagggttccagcttagccagccgtatcattgagaggttatcatcaatggcccactctgtgattggatttatccaacgcatgttctcggaggggagccataaaccggtggtgacaccagcactcacgcctgcacaaatgccaagccctacgtctttcagtgatagtatcaagcaacttgctgctgagacgctgccaaaatacatgcagcagttgagtagcttggatgcagagacgctgcagaaaaatcatgaccagttcgccacgggcagcggccctcttcgtggcagtatcactcaatgccaagggctgatgcagttttgtggtggggaattgcaagctgaggccagtgccattttaaacacgcctgtttgtggtattcccttctcgcagtggggaactgttggtggggcggccagcgcgtacgtcgccagtggcgttgatctaacgcaggcagcaaatgagatcaaagggctggggcaacagatgcagcaattactgtcattgatgtgatatggataaaaacaagggggtagtgtttccccctttttctatcaatattgcgaatatcttcgtccctgatctttcaggggcgaatcgttttttagcatgctcattgttagaatttctgacttatctctcttctgtattactactcatactctggaaaatcctgagcatttatatctatggattgatgcagcactcgag

SEQ ID NO: 3 - protein, 462 aa, Shigella flexneri
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMSIEIKMISPIKNIKNVFPINTANTEYIVRNIYPRVEHGYFNESPNIYDKKYISGITRSMAQLKIEEFINEKSRRLNYMKTMYSPCPEDFQPISRDEASTPEGSWLTVISGKRPMGQFSVDSLYHPDLHALCELPEISCKIFPKENSDFLYIIVVFRNDSPQGELRANRFIELYDIKREIMQVLRDESPELKSIKSEIIIAREMGELFSYASEEIDSYIKQMNDRLSQIKARMPVT

SEQ ID NO: 4 - DNA, 1386 bp, Shigella flexneri
atgagcccgattctgggctattggaaaattaaaggcctggtgcagccgacccgcctgctgctggaatatctggaagaaaaatatgaagaacatctgtatgaacgcgatgaaggcgataaatggcgcaacaaaaaatttgaactgggcctggaatttccgaacctgccgtattatattgatggcgatgtgaaactgacccagagcatggcgattattcgctatattgcggataaacataacatgctgggcggctgcccgaaagaacgcgcggaaattagcatgctggaaggcgcggtgctggatattcgctatggcgtgagccgcattgcgtatagcaaagattttgaaaccctgaaagtggattttctgagcaaactgccggaaatgctgaaaatgtttgaagatcgcctgtgccataaaacctatctgaacggcgatcatgtgacccatccggattttatgctgtatgatgcgctggatgtggtgctgtatatggatccgatgtgcctggatgcgtttccgaaactggtgtgctttaaaaaacgcattgaagcgattccgcagattgataaatatctgaaaagcagcaaatatattgcgtggccgctgcagggctggcaggcgacctttggcggcggcgatcatccgccgaaaagcgatctggtgccgcgcggcagcatgagcattgaaattaaaatgattagcccgattaaaaacattaaaaacgtgtttccgattaacaccgcgaacaccgaatatattgtgcgcaacatttatccgcgcgtggaacatggctattttaacgaaagcccgaacatttatgataaaaaatatattagcggcattacccgcagcatggcgcagctgaaaattgaagaatttattaacgaaaaaagccgccgcctgaactatatgaaaaccatgtatagcccgtgcccggaagattttcagccgattagccgcgatgaagcgagcaccccggaaggcagctggctgaccgtgattagcggcaaacgcccgatgggccagtttagcgtggatagcctgtatcatccggatctgcatgcgctgtgcgaactgccggaaattagctgcaaaatttttccgaaagaaaacagcgattttctgtatattattgtggtgtttcgcaacgatagcccgcagggcgaactgcgcgcgaaccgctttattgaactgtatgatattaaacgcgaaattatgcaggtgctgcgcgatgaaagcccggaactgaaaagcattaaaagcgaaattattattgcgcgcgaaatgggcgaactgtttagctatgcgagcgaagaaattgatagctatattaaacagatgaacgatcgcctgagccagattaaagcgcgcatgccggtgacc

SEQ ID NO: 5 - protein, 330 aa, Citrobacter rodentium
MKIPSLQSNFNFSAPAGYSAPIAPNRAENAYADYVLDIGKRIPLSAADLSNVYESVIRAVHDSRSRLIDQHTVDMIGNTVLDALSRSQTFRDAVSYGIHNEKVHIGCIKYRNEYELNEESSVKIDDIQSLTCNELYEYDVGQEPIFPICEAGENDNEEPYVSFSVAPDTDSYEMPSWQEGLIHEIIHHVTGSSDPSGDSNIELGPTEILARRVAQELGWSVPDFKGYAEPEREAHLRLRNLNALRQAAMRHEENERAFFERLGTISDRYEASPDFTEYSAVSNIGYGFIQQHDFPGLAINDNLQDANQIQLYHGAPYIFTFGDVDKHNQR

SEQ ID NO: 6 - DNA, 1464 bp, Citrobacter rodentium
gaattcatccggtcaaacggcttctttttgcaggaaaggaatatgagttaaaggtcattgatgaaaaaacgcctattatcctgctctacttgagggattgtcttttatgcacagaagagtagatgctcatccatattatgatggtttaggtaaagggataaagaaatattttgattttactcaattacatgattacaatcatttttatgactttattgagtttaaacatccaaatattattatgaacacaagtcagtatacaggcagttcatggtaaatggtttttacatagtttattctgttgtaataaatgattagcatggtattaggtatcaacatgaaaattccctcactccagcccagcttcaactttttcgccccagcaggatactctgctgccgttgctcccaatcgttcggacaatgcctatgctgattacgtattggatataggcaagcgaataccactttccgcggaagatttaggcaacctatatgaaaatgtcattcgcgccgttcgtgacagccgtagcaagctcatagatcagcatacggtcgatatgattggtaacactatacttgatgctttgagccgatcacaaacctttcgtgatgccgtaagctatggcattcataataaggaggtacacattggttgcattaaatacagaaacgaatacgagctcaacggagaatcccccgtcaaagttgatgatattcaatcactaacctgtaccgaattatatgaatacgatgtcgggcaagaaccaattttacccatttgcgaggcaggagaaaacgataacgaagagccttatgtcagttttagtgttgcgccagatactgactcttatgagatgccatcgtggcaggaagggctgattcacgagattattcatcatgtgactggagctagcgatccgtctggagatagtaatatagagctaggacccacggagattctcgcacgtcgtgtcgctcaagagctgggatggactgtccccgacttcataggatatgcagagccagatcgtgaagctcatcttaggggacgtaacctgaatgcccttcgacaggcggccatgcgacatgaagataatgagaggactttcttcgaaaggctgggtatgatcagtgatcgatatgaggcgagtcctgatttcacagagtattccgctgtgtctaacatagaatatggatttatccagcaacatgattttcccgggttggctatcgacgataatttacaggatgcaaatcagatccaactctatcatggagcaccttatatctttacattcggggatgtggacaaacacaatcagcgctgacgcgtctttgcagcgacacaaggctactactcttgcattttaacggagttgatgatggaaaatcgtgcaaccttgtatgtaaaggcgaaaaaccaaattttacggtagtaagtgagcctggcgggaatggtacc

SEQ ID NO: 7 - protein, 288 aa, Yersinia pseudotuberculosis
MIGPISQINISGGLSEKETSSLISNEELKNIITQLETDISDGSWFHKNYSRMDVEVMPALVIQANNKYPEMNLNLVTSPLDLSIEIKNVIENGVRSSRFIINMGEGGIHFSVIDYKHINGKTSLILFEPANFNSMGPAMLAIRTKTAIERYQLPDCHFSMVEMDIQRSSSECGIFSFALAKKLYIERDSLLKIHEDNIKGILSDGENPLPHDKLDPYLPVTFYKHTQGKKRLNEYLNTNPQGVGTVVNKKNETIVNRFDNNKSIVDGKELSVSVHKKRIAEYKTLLKV

SEQ ID NO: 8 - DNA, 867 bp, Yersinia pseudotuberculosis
atgatcggaccaatatcacaaataaatatctccggtggcttatcagaaaaagagaccagttctttaatcagtaatgaagagcttaaaaatatcataacacagttggaaactgatatatcggatggatcctggttccataaaaattattcacgtatggatgtagaagtcatgcccgcattggtaatccaggcgaacaataaatatccggaaatgaatcttaatcttgttacatctccattggacctttcaatagaaataaaaaacgtcatagaaaatggagttagatcttcccgcttcataattaacatgggggaaggtggaatacatttcagtgtaattgattacaaacatataaatgggaaaacatctctgatattgtttgaaccagcaaactttaacagtatggggccagcgatgctggcaataaggacaaaaacggctattgaacgttatcaattacctgattgccatttctccatggtggaaatggatattcagcgaagctcatctgaatgtggtatttttagttttgcactggcaaaaaaactttacatcgagagagatagcctgttgaaaatacatgaagataatataaaaggtatattaagtgatggtgaaaatcctttaccccacgataagttggacccgtatctcccggtaactttttacaaacatactcaaggtaaaaaacgtcttaatgaatatttaaatactaacccgcagggagttggtactgttgttaacaaaaaaaatgaaaccatcgttaatagatttgataacaataaatccattgtagatggaaaggaattatcagtttcggtacataaaaagagaatagctgaatataaaacacttctcaaagtataa

SEQ ID NO: 9 - protein, 336 aa, Escherichia coli
MIPPLNRYVPALSKNELVKTVTNRDIQFTSFNGKDYPLCFLDEKTPLLFQWFERNPARFGKNDIPIINTEKNPYLNNIIKAATIEKERLIGIFVDGDFFPGQKDAFSKLEYDYENIKVIYRNDIDFSMYDKKLSEIYMENISKQESMPEEKRDCHLLQLLKKELSDIQEGNDSLIKSYLLDKGHGWFDFYRNMAMLKAGQLFLEADKVGCYDLSTNSGCIYLDADMIITEKLGGIYIPDGIAVHVERIDGRASMENGIIAVDRNNHPALLAGLEIMHTKFDADPYSDGVCNGIRKHFNYSLNEDYNSFCDFIEFKHDNIIMNTSQFTQSSWARHVQ

SEQ ID NO: 10 - DNA, 540 bp, Escherichia coli
atgttatctccattaaatgttcttcaatttaatttcagaggagagaccgctttatcagatagtgctcctctccagactgtttcctttgctggaaaagattattctatggaacccattgatgaaaaaacacccattctttttcagtggtttgaagcaaggccagagcgatacggaaaaggtgaagtaccgatattgaataccaaagagcatccgtatttgagcaatattataaatgctgcaaaaatagaaaatgagcgcgtaataggagtactggtagacggagactttacttatgagcaaagaaaagaatttctcagtcttgaagatgaacatcaaaatataaagataatatatcgggaaaatgttgatttcagtatgtatgataaaaaactgtctgatatttatcttgaaaatattcatgaacaagaatcatatccagcgagtgagagagataattatctgttaggcttattaagagaagagttaaaaaatattccatacggaaaggactctttgattgaatcatatgcagaaaaaaga

SEQ ID NO: 11 - protein, 545 aa, Shigella flexneri
MLPINNNFSLPQNSFYNTISGTYADYFSAWDKWEKQALPGEERDEAVSRLKECLINNSDELRLDRLNLSSLPDNLPAQITLLNVSYNQLTNLPELPVTLKKLYSASNKLSELPVLPPALESLQVQHNELENLPALPDSLLTMNISYNEIVSLPSLPQALKNLRATRNFLTELPAFSEGNNPVVREYFFDRNQISHIPESILNLRNECSIHISDNPLSSHALQALQRLTSSPDYHGPRIYFSMSDGQQNTLHRPLADAVTAWFPENKQSDVSQIWHAFEHEEHANTFSAFLDRLSDTVSARNTSGFREQVAAWLEKLSASAELRQQSFAVAADATESCEDRVALTWNNLRKTLLVHQASEGLFDNDTGALLSLGREMFRLEILEDIARDKVRTLHFVDEIEVYLAFQTMLAEKLQLSTAVKEMRFYGVSGVTANDLRTAEAMVRSREENEFTDWFSLWGPWHAVLKRTEADRWAQAEEQKYEMLENEYPQRVADRLKASGLSGDADAEREAGAQVMRETEQQIYRQLTDEVLALRLSENGSQLHHS

SEQ ID NO: 12 - DNA, 1822 bp, Shigella flexneri
gttaactgaaacagtatcgtttttttacagccaattttgtttatccttattataataaaaaagtgctgaagttcatttcatggaatgaacttcataaaaactcctacttatttcttttaacaaagccatttgtccaccggctttaactggatgcccatcatgttaccgataaataataacttttcattgccccaaaattctttttataacactatttccggtacatatgctgattacttttcagcatgggataaatgggaaaaacaagcgctccccggtgaagagcgtgatgaggctgtctcccgacttaaagaatgtcttatcaataattccgatgaacttcgactggaccgtttaaatctgtcctcgctacctgacaacttaccagctcagataacgctgctcaatgtatcatataatcaattaactaacctacctgaactgcctgttacgctaaaaaaattatattccgccagcaataaattatcagaattgcccgtgctacctcctgcgctggagtcacttcaggtacaacacaatgagctggaaaacctgccagctttacccgattcgttattgactatgaatatcagctataacgaaatagtctccttaccatcgctcccacaggctcttaaaaatctcagagcgacccgtaatttcctcactgagctaccagcattttctgagggaaataatcccgttgtcagagagtatttttttgatagaaatcagataagtcatatcccggaaagcattcttaatctgaggaatgaatgttcaatacatattagtgataacccattatcatcccatgctctgcaagccctgcaaagattaacctcttcgccggactaccacggcccacggatttacttctccatgagtgacggacaacagaatacactccatcgccccctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggatattgcccgggataaagtcagaactctccattttgtggatgagatagaagtctacctggccttccagaccatgctcgcagagaaacttcagctctccactgccgtgaaggaaatgcgtttctatggcgtgtcgggagtgacagcaaatgacctccgcactgccgaagccatggtcagaagccgtgaagagaatgaatttacggactggttctccctctggggaccatggcatgctgtactgaagcgtacggaagctgaccgctgggcgcaggcagaagagcagaaatatgagatgctggagaatgagtaccctcagagggtggctgaccggctgaaagcatcaggtctgagcggtgatgcggatgcggagagggaagccggtgcacaggtgatgcgtgagactgaacagcagatttaccgtcagctgactgacgaggtactggccctgcgattgtctgaaaacggctcacaactgcaccattcataatcacatcgcataaaactgtcagcgc

SEQ ID NO: 13 - protein, 574 aa, Shigella flexneri
MKPINNHSFFRSLCGLSCISRLSVEEQCTRDYHRIWDDWAREGTTTENRIQAVRLLKICLDTREPVLNLSLLKLRSLPPLPLHIRELNISNNELISLPENSPLLTELHVNGNNLNILPTLPSQLIKLNISFNRNLSCLPSLPPYLQSLSARFNSLETLPELPSTLTILRIEGNRLTVLPELPHRLQELFVSGNRLQELPEFPQSLKYLKVGENQLRRLSRLPQELLALDVSNNLLTSLPENIITLPICTNVNISGNPLSTHVLQSLQRLTSSPDYHGPQIYFSMSDGQQNTLHRPLADAVTAWFPENKQSDVSQIWHAFEHEEHANTFSAFLDRLSDTVSARNTSGFREQVAAWLEKLSASAELRQQSFAVAADATESCEDRVALTWNNLRKTLLVHQASEGLFDNDTGALLSLGREMFRLEILEDIARDKVRTLHFVDEIEVYLAFQTMLAEKLQLSTAVKEMRFYGVSGVTANDLRTAEAMVRSREENEFTDWFSLWGPWHAVLKRTEADRWAQAEEQKYEMLENEYSQRVADRLKASGLSGDADAEREAGAQVMRETEQQIYRQLTDEVLA

SEQ ID NO: 14 - DNA, 1725 bp, Shigella flexneri
atgaaaccgatcaacaatcattctttttttcgttccctttgtggcttatcatgtatatctcgtttatcggtagaagaacagtgtaccagagattaccaccgcatctgggatgactgggctagggaaggaacaacaacagaaaatcgcatccaggcggttcgattattgaaaatatgtctggatacccgggagcctgttctcaatttaagcttactgaaactacgttctttaccaccactccctttgcatatacgtgaacttaatatttccaacaatgagttaatctccctacctgaaaattctccgcttttgacagaacttcatgtaaatggtaacaacttgaatatactcccgacacttccatctcaactgattaagcttaatatttcattcaatcgaaatttgtcatgtctgccatcattaccaccatatttacaatcactctcggcacgttttaatagtctggagacgttaccagagcttccatcaacgctaacaatattacgtattgaaggtaatcgccttactgtcttgcctgaattgcctcatagactacaagaactctttgtttccggcaacagactacaggaactaccagaatttcctcagagcttaaaatatttgaaggtaggtgaaaatcaactacgcagattatccagattaccgcaagaactattggcactggatgtttccaataacctactaacttcattacccgaaaatataatcacattgcccatttgtacgaatgttaacatttcagggaatccattgtcgactcacgttctgcaatccctgcaaagattaacctcttcgccggactaccacggcccgcagatttacttctccatgagtgacggacaacagaatacactccatcgccccctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggatattgcccgggataaagtcagaactctccattttgtggatgagatagaagtctacctggccttccagaccatgctcgcagagaaacttcagctctccactgccgtgaaggaaatgcgtttctatggcgtgtcgggagtgacagcaaatgacctccgcactgccgaagctatggtcagaagccgtgaagagaatgaatttacggactggttctccctctggggaccatggcatgctgtactgaagcgtacggaagctgaccgctgggcgcaggcagaagagcagaagtatgagatgctggagaatgagtactctcagagggtggctgaccggctgaaagcatcaggtctgagcggtgatgcggatgcggagagggaagccggtgcacaggtgatgcgtgagactgaacagcagatttaccgtcagttgactgacgaggtactggcctga

SEQ ID NO: 15 - protein, 367 aa, Yersinia pestis
MFITPRNVSNTFLQEPLRHSSDLTEMPVEAENVKSKTEYYNAWAVWERNAPPGNGEQREMAVSRLRDCLDRQAHELELNNLGLSSLPELPPHLESLVASCNSLTELPELPQSLKSLQVDNNNLKALSDLPPSLEFLAAGNNQLEELPELQNSSFLKIIDVDNNSLKKLPDLPPSLEFLAAGNNQLEELSELQNLPFLTEIHADNNSLKTLPDLPPSLKTLNVRENYLTDLPELPQSLTFLDVSDNIFSGLSELPPNLYYLDASSNGIRSLCDLPPSLVELDVRDNQLIELPALPPHLERLIASLNHLAEVPELPQNLKQLHVEHNALREFPDIPESVEDLRMDSERVTDTYEFAHETTDKLEDDVFE

SEQ ID NO: 16 - DNA, 1104 bp, Yersinia pestis
atgtttataactccaagaaatgtatctaatacttttttgcaagaaccattacgtcattcttctgatttaactgagatgccggttgaagcagaaaatgttaaatctaagactgaatattataatgcatgggcggtatgggaacgaaatgcccctccggggaatggtgaacagagggaaatggcggtttcaaggttacgcgattgcctggaccgacaagcccatgagctagaactaaataatctggggctgagttctttgccggaattacctccgcatttagagagtttagtggcgtcatgtaattctcttacagaattaccggaattgccgcagagcctgaaatcacttcaagttgataataacaatctgaaggcattatccgatttacctccttcactggaatttcttgctgctggtaataatcagctggaagaattgccagagttgcaaaactcgtccttcttgaaaattattgatgttgataacaattcactgaaaaaactacctgatttacctccttcactggaatttcttgctgctggtaataatcagctggaagaattgtcagagttacaaaacttgccattcttgactgagattcatgctgataacaattcactgaaaacattacccgatttacccccttccctgaaaacacttaatgtcagagaaaattatttaactgatctgccagaattaccgcagagtttaaccttcttagatgtttctgataatattttttctggattatcggaattgccaccaaacttgtattatctcgatgcatccagcaatggaataagatccttatgcgatttacccccttcactggtagaacttgatgtcagagataatcagttgatcgaactgccagcgttacctccacacttagaacgtttaatcgcttcacttaatcatcttgctgaagtacctgaattgccgcaaaacctgaaacagctccacgtagagcacaacgctctaagagagtttcccgatatacctgagtcagtggaagatcttcggatggactctgaacgtgtaactgatacatatgaatttgctcatgagactacagacaaacttgaagatgatgtatttgagtag

SEQ ID NO: 17 - protein, 86 aa, Yersinia pestis
MFINPRNVSNTFLQEPLRHSSNLTEMPVEAENVKSKTEYYNAWSEWERNAPPGNGEQREMAVSRLRDCLDRQAHELELNNLGLSSL

SEQ ID NO: 18 - DNA, 258 bp, Yersinia pestis
atgttcatcactccacgcaatgtatctaacacctttctgcaggaaccgctgcgtcattctagcgacctgaccgaaatgccagttgaagcggagaacgtgaaatctaagactgaatactacaacgcgtgggcagtatgggagcgcaatgcaccaccaggtaacggtgaacagcgtgaaatggcagtaagccgtctgcgtgattgcctggatcgccaggctcacgagctggagctgaacaacctgggtctgtctagcctg

SEQ ID NO: 19 - protein, 131 aa, Yersinia pestis
MFINPRNVSNTFLQEPLRHSSNLTEMPVEAENVKSKTEYYNAWSEWERNAPPGNGEQREMAVSRLRDCLDRQAHELELNNLGLSSLPELPPHLESLVASCNSLTELPELPQSLKSLLVDNNNLKALSDLPP

SEQ ID NO: 20 - DNA, 393 bp, Yersinia pestis
atgtttattaacccgcgcaacgtgagcaacacctttctgcaggaaccgctgcgccatagcagcaacctgaccgaaatgccggtggaagcggaaaacgtgaaaagcaaaaccgaatattataacgcgtggagcgaatgggaacgcaacgcgccgccgggcaacggcgaacagcgcgaaatggcggtgagccgcctgcgcgattgcctggatcgccaggcgcatgaactggaactgaacaacctgggcctgagcagcctgccggaactgccgccgcatctggaaagcctggtggcgagctgcaacagcctgaccgaactgccggaactgccgcagagcctgaaaagcctgctggtggataacaacaacctgaaagcgctgagcgatctgccgccg

SEQ ID NO: 21 - protein, 565 aa, Shigella flexneri
MFSVNNTHSSVSCSPSINSNSTSNEHYLRILTEWEKNSSPGEERGIAFNRLSQCFQNQEAVLNLSDLNLTSLPELPKHISALIVENNKLTSLPKLPAFLKELNADNNRLSVIPELPESLTTLSVRSNQLENLPVLPNHLTSLFVENNRLYNLPALPEKLKFLHVYYNRLTTLPDLPDKLEILCAQRNNLVTFPQFSDRNNIRQKEYYFHFNQITTLPESFSQLDSSYRINISGNPLSTRVLQSLQRLTSSPDYHGPQIYFSMSDGQQNTLHRPLADAVTAWFPENKQSDVSQIWHAFEHEEHANTFSAFLDRLSDTVSARNTSGFREQVAAWLEKLSASAELRQQSFAVAADATESCEDRVALTWNNLRKTLLVHQASEGLFDNDTGALLSLGREMFRLEILEDIARDKVRTLHFVDEIEVYLAFQTMLAEKLQLSTAVKEMRFYGVSGVTANDLRTAEAMVRSREENEFTDWFSLWGPWHAVLKRTEADRWAQAEEQKYEMLENEYSQRVADRLKASGLSGDADAEREAGAQVMRETEQQIYRQLTDEVLALRLSENGSRLHHS

SEQ ID NO: 22 - DNA, 1698 bp, Shigella flexneri
atgttctctgtaaataatacacactcatcagtttcttgctccccctctattaactcaaactcaaccagtaatgaacattatctgagaatcctgactgaatgggaaaagaactcttctcccggggaagagcgaggcattgcttttaacagactctcccagtgctttcagaatcaagaagcagtattaaatttatcagacctaaatttgacgtctcttcccgaattaccaaagcatatttctgctttgattgtagaaaataataaattaacatcattgccaaagctgcctgcatttcttaaagaacttaatgctgataataacaggctttctgtgataccagaacttcctgagtcattaacaactttaagtgttcgttctaatcaactggaaaaccttcctgttttgccaaaccatttaacatcattatttgttgaaaataacaggctatataacttaccggctcttcccgaaaaattgaaatttttacatgtttattataacaggctgacaacattacccgacttaccggataaactggaaattctctgtgctcagcgcaataatctggttacttttcctcaattttctgatagaaacaatatcagacaaaaggaatattattttcattttaatcagataaccactcttccggagagtttttcacaattagattcaagttacaggattaatatttcagggaatccattgtcgactcgcgttctgcaatccctgcaaagattaacctcttcgccggactaccacggcccgcagatttacttctccatgagtgacggacaacagaatacactccatcgccccctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggacattgcccgggataaagtcagaactctccattttgtggatgagatagaagtctacctggccttccagaccatgctcgcagagaaacttcagctctccactgccgtgaaggaaatgcgtttctatggcgtgtcgggagtgacagcaaatgacctccgcactgccgaagctatggtcagaagccgtgaagagaatgaatttacggactggttctccctctggggaccatggcatgctgtactgaagcgtacggaagctgaccgctgggcgcaggcagaagagcagaagtatgagatgctggagaatgagtactctcagagggtggctgaccggctgaaagcatcaggtctgagcggtgatgcggatgcggagagggaagccggtgcacaggtgatgcgtgagactgaacagcagatttaccgtcagctgactgacgaggtactggccctgcgattgtctgaaaacggctcacgactgcaccattcataa

SEQ ID NO: 23 - protein, 379 aa, Artificial Sequence
  Description of Artificial Sequence Synthetic polypeptide
MFITPRNVSNTFLQEPLRHSSDLTEMPVEAENVKSKTEYYNAWAVWERNAPPGNGEQREMAVSRLRDCLDRQAHELELNNLGLSSLPELPPHLESLVASCNSLTELPELPQSLKSLQVDNNNLKALSDLPPSLEFLAAGNNQLEELPELQNSSFLKIIDMSIEIKMISPIKNIKNVFPINTANTEYIVRNIYPRVEHGYFNESPNIYDKKYISGITRSMAQLKIEEFINEKSRRLNYMKTMYSPCPEDFQPISRDEASTPEGSWLTVISGKRPMGQFSVDSLYHPDLHALCELPEISCKIFPKENSDFLYIIVVFRNDSPQGELRANRFIELYDIKREIMQVLRDESPELKSIKSEIIIAREMGELFSYASEEIDSYIK

SEQ ID NO: 24 - protein, 345 aa, Artificial Sequence
  Description of Artificial Sequence Synthetic polypeptide
MFITPRNVSNTFLQEPLRHSSDLTEMPVEAENVKSKTEYYNAWAVWERNAPPGNGEQREMAVSRLRDCLDRQAHELELNNLGLSSLPELPPHLESLVASCNSLTELPELPQSLKSLQVDNNNLKALSDLPPSLEFLAAGNNQLEELPELQNSSFLKIIDKIPSLQSNFNFSAPAGYSAPIAPNRAENAYADYVLDIGKRIPLSAADLSNVYESVIRAVHDSRSRLIDQHTVDMIGNTVLDALSRSQTFRDAVSYGIHNEKVHIGCIKYRNEYELNEESSVKIDDIQSLTCNELYEYDVGQEPIFPICEAGENDNEEPYVSFSVAPDTDSYEMPSWQEGLIHEIIH

SEQ ID NO: 25 - protein, 30 aa, Artificial Sequence
  Description of Artificial Sequence Synthetic polypeptide
  MISC_FEATURE (1)..(30): This sequence may encompass 1-6 “Gly Gly Gly Gly Ser” repeating units
GGGGSGGGGSGGGGSGGGGSGGGGSGGGGS

SEQ ID NO: 26 - protein, 25 aa, Artificial Sequence
  Description of Artificial Sequence Synthetic peptide
  MOD_RES (1)..(4): Thr or Ser
  MOD_RES (6)..(9): Thr or Ser
  MOD_RES (11)..(14): Thr or Ser
  MOD_RES (16)..(19): Thr or Ser
  MOD_RES (21)..(24): Thr or Ser
  MISC_FEATURE (1)..(25): This sequence may encompass 1-5 “Xaa Xaa Xaa Xaa Gly” repeating units, wherein Xaa may be Thr or Ser
  See specification as filed for detailed description of substitutions and preferred embodiments
XXXXGXXXXGXXXXGXXXXGXXXXG

SEQ ID NO: 27 - protein, 5 aa, Artificial Sequence
  Description of Artificial Sequence Synthetic peptide
SSSSG

SEQ ID NO: 28 - protein, 5 aa, Artificial Sequence
  Description of Artificial Sequence Synthetic peptide
PAPAP

SEQ ID NO: 29 - protein, 15 aa, Artificial Sequence
  Description of Artificial Sequence Synthetic peptide
  MISC_FEATURE (1)..(14): This region may encompass 2-7 “Pro Thr” repeating units
PTPTPTPTPTPTPTP

SEQ ID NO: 30 - protein, 27 aa, Artificial Sequence
  Description of Artificial Sequence Synthetic peptide
  MISC_FEATURE (2)..(26): This region may encompass 1-5 “Glu Ala Ala Ala Lys” repeating units
AEAAAKEAAAKEAAAKEAAAKEAAAKA